(19) United States
(12) Patent Application Publication
(10) Pub. No.: US 2026/0013747 A1
(43) Pub. Date: Jan. 15, 2026

(54) LIGHT SOURCE DEVICE AND MEASUREMENT SYSTEM

(71) Applicants: Katsuyuki SAKURANO, Hyogo (JP); Masaaki YOSHIDA, Osaka (JP); Naoto JIKUTANI, Miyagi (JP); Kazuhiro HARASAKA, Miyagi (JP)

(72) Inventors: Katsuyuki SAKURANO, Hyogo (JP); Masaaki YOSHIDA, Osaka (JP); Naoto JIKUTANI, Miyagi (JP); Kazuhiro HARASAKA, Miyagi (JP)

(21) Appl. No.: 18/992,185

(22) PCT Filed: Jul. 20, 2023
(86) PCT No.: PCT/IB2023/057380

§ 371 Date: Jan. 8, 2025

(30) Foreign Application Priority Data
Jul. 29, 2022 (JP) 2022-121208
Jun. 12, 2023 (JP) 2023-096438

(51) Int. Cl. A61B 5/026; A61B 5/145; A61B 5/1455; A61B 5/16

(52) U.S. Cl. CPC A61B 5/0261; A61B 5/14546; A61B 5/1455; A61B 5/165

(57) ABSTRACT

A light source device includes; a surface emitting laser to emit a light beam to a subject; and circuitry to drive the surface emitting laser to emit a light having a time width, a light intensity of which is 1/e² with respect to a peak of the light intensity, of 200 picoseconds or less.

### TECHNICAL FIELD

The present disclosure relates to a light source device and a measurement system.

### BACKGROUND ART

In the related art, near-infrared spectroscopy (NIRS) is a technique to observe a blood flow state inside a subject from an optical signal returned after infrared light incident in the subject. Since near-infrared light has a higher transmissivity to a living body, blood flow or oxygen metabolism change of the living tissue can be measured. The research to estimates the mental state such as the emotion based on information of skin blood flow and brain blood flow obtained by NIRS has been also carried out.

In a typically NIRS, a continuous wave (CW) method is the mainstream, but a time domain (TD) method has been examined. In the CW method, a light beam passed through a shallow portion of the subject and returned is mainly detected. By contrast, in the TD method, a light beam passed through a deep portion of the subject and returned can be detected. Thus, the TD method is effective to obtain information on the inside of the living body.

In the TD method, a pulse light beam is used instead of a continuous light beam. In PLT 1, a single light irradiation means to emit a pulse light beam to a scattering and absorption body from a predetermined light irradiation position for non-invasive measurement of internal information on the scattering and absorption body and N (N is integer greater than two) measurement modules including each of multiple light detection means to detect the light beams emitted from the single light emission means and propagating inside the scattering and absorption body at each predetermined detection positions are disclosed.

### CITATION LIST

### Patent Literature

### [PTL 1]

Japanese Patent No. 4167144

### Non Patent Literature

### [NPL1]

Kobayashi, Soichi, et al. “Direct frequency modulation in AlGaAs semiconductor lasers. “IEEE Transactions on Microwave Theory and Techniques 30.4 (1982): 428-441

### [NPL 2]

Ban, Han, Barrett, Geoffrey, Borisevich, Alex, Chaturvedi, Ashutosh, Dahle, Jacob, et al.; “Kernel Flow: a high channel count scalable TD-fNIRS system” Proc. Of SPIE Vol. 11663 116630B-19

### SUMMARY OF INVENTION

### Technical Problem

A short pulse light source that can be used in measurement of inside of a subject that is a scattering body or a transparent body has not sufficiently been examined.

An aim of the present invention is to provide a short pulse light source that can measure the inside of the subject of a scattering body or a transparent body.

### Solution to Problem

A light source device includes; a surface emitting laser to emit a light beam to a subject; and circuitry to drive the surface emitting laser to emit a light having a time width, a light intensity of which is 1/e² with respect to a peak of the light intensity, of 200 picoseconds or less.

Further, an embodiment of the present disclosure provides a measurement system includes: the light source device described above; a detector to detect multiple scattering light beams propagating inside the subject and output a detection signal including information of a detection timing, the circuitry to: input and process the detection signal; and control the surface emitting laser to emit pulse light beams to an interior of the subject multiple times, and the pulse light beam having a time width, a light intensity of which is 1/e² with respect to the peak, of 200 picoseconds or less.

### Advantageous Effects of Invention

According to the embodiments of the present invention, a short pulse light source that can preferably measure the inside of the subject that is a scattering body or a transparent body can be provided.

### BRIEF DESCRIPTION OF DRAWINGS

The accompanying drawings are intended to depict example embodiments of the present invention and should not be interpreted to limit the scope thereof. The accompanying drawings are not to be considered as drawn to scale unless explicitly noted. Also, identical or similar reference numerals designate identical or similar components throughout the several views.

FIG. 1 is a diagram of the present embodiment;

FIG. 2 is a block diagram of the present embodiment;

FIG. 3 is a flowchart according to the present embodiment;

FIG. 4A is a graph of an ideal projection light waveform and ideal reception light waveforms (a shallow position and a deep position);

FIG. 4B is a graph of ideal projection light waveforms (an ideal waveform and a comparative waveform);

FIG. 4C is a graph of reception light waveforms (a deep position and a shallow position) in a case where the comparative waveform in FIG. 4B is used;

FIG. 5 is a diagram of a configuration of a surface emitting laser;

FIG. 6 is a diagram of an oxidation confinement portion of a gamma-switching type surface emitting laser;

FIG. 7 is a diagram of an oxidation confinement portion as a comparative example;

FIG. 8 is a diagram of a circuit for measurement;

FIG. 9A is a graph of a measurement waveform as a comparative example;

FIG. 9B is a graph of a measurement waveform as a comparative example;

FIG. 9C is a graph of a measurement waveform as a comparative example;

FIG. 10A is a graph of a measurement waveform of the gamma-switching type surface emitting laser;

FIG. 10B is a graph of a measurement waveform of the gamma-switching type surface emitting laser;

FIG. 10C is a graph of a measurement waveform of the gamma-switching type surface emitting laser;

FIG. 11A is a diagram of an operation principle (a refractive index distribution by the structure) for the gamma-switching type surface emitting laser;

FIG. 11B is a diagram of an operation principle (a refractive index distribution by the structure) for the gamma-switching type surface emitting laser;

FIG. 12A is a diagram of an operation principle (a refractive index change) for the gamma-switching type surface emitting laser;

FIG. 12B is a diagram of an operation principle (a refractive index change) for the gamma-switching type surface emitting laser;

FIG. 13 is a graph of a measurement waveform of the gamma-switching type surface emitting laser;

FIG. 14A is a diagram of a light source module;

FIG. 14B is a diagram of a light detection module;

FIG. 15 is a diagram of a light source module and a light detection module;

FIG. 16 is a diagram of an installation example according to the present embodiment; and

FIG. 17 is a diagram of a measurement portion of the present embodiment.

### DESCRIPTION OF EMBODIMENTS

The terminology used herein is for the purpose of describing particular embodiments only and is not intended to be limiting of the present invention. As used herein, the singular forms “a,” “an,” and “the” are intended to include the plural forms as well, unless the context clearly indicates otherwise.

In describing embodiments illustrated in the drawings, specific terminology is employed for the sake of clarity. However, the disclosure of this specification is not intended to be limited to the specific terminology so selected and it is to be understood that each specific element includes all technical equivalents that have a similar function, operate in a similar manner, and achieve a similar result.

### DESCRIPTION OF EMBODIMENTS

Embodiments of the present disclosure will be described with reference to the drawings. In the description of the drawings, the same components are denoted by the same reference numerals, and overlapping description will be omitted.

### Embodiment

Embodiments of the present invention will be described. In the present embodiment, near-infrared spectroscopy (NIRS) that is a technique to observe the blood flow state inside a subject from an optical signal returned after infrared light incident in the subject has following features.

Since a pulse laser beam, which is a single pulse, having the time width of 200 picoseconds (psec) or less in which an light intensity becomes 1/e² of a peak intensity and no skirts is used as a light source to project a light beam, an optical signal returned after injection of the pulse laser beam into a target medium is not affected by the skirts of the pulse laser beam injected and can be measured even if the optical signal is low level. Herein, in the typical short pulse light source, a full width at half maximum (FWHM) that is a time width in which an intensity becomes ½ of a peak intensity is mainly used. As described below, the inventors found that only a small FWHM is insufficient for features of the short pulse light source. In the present disclosure, in a projection light waveform, a time width in which an light intensity becomes 1/e² of a peak intensity is denoted as a 1/e² pulse width in the following description.

The features of the present embodiment described above will be described in detail with reference to the drawings.

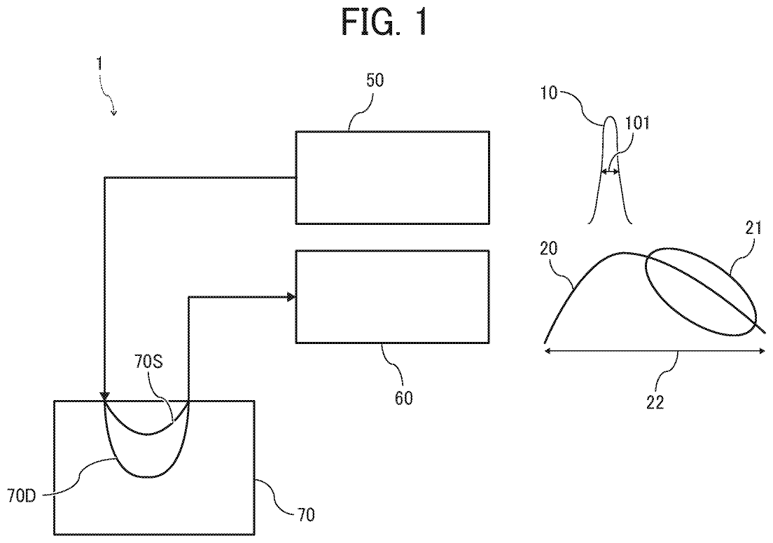

FIG. 1 is a diagram of an overall configuration of a time domain near infrared spectroscopy (TD-NIRS) system 1 as an example of a measurement system.

A light source module 50 as an example of a light projection means includes a laser light source 501 and emits an irradiation beam (a laser light beam) to a target medium 70. In a projection light waveform 10 from the light source module 50, the intensity of the light is represented in the vertical axis, the time is represented in the horizontal axis, and a time width during which the light exists is represented by a time width 101. In a feature of the projection light waveform 10 according to the present embodiment, the time width is 200 psec or less in which the light intensity becomes 1/e² of the peak intensity. As another feature, the projection light waveform 10 is a short pulse without the skirts, and time course in which the light intensity becomes 1/1,000 of the peak intensity is two nanoseconds (nsec) or less. A pulse shape will be described in detail below.

In some embodiments, a light source device includes; a surface emitting laser to emit a light beam to a subject; and circuitry to drive the surface emitting laser to emit a light having a time width, a light intensity of which is 1/e² with respect to a peak of the light intensity, of 200 picoseconds or less.

The laser light source 501 oscillates repeatedly every several tens of nsec, but oscillates only one pulse during the period. The laser light source 501 does not emit excess light after the emission of one pulse. Specifically, by using a gamma-switching type vertical cavity surface emitting laser (VCSEL), an ideal short-pulse laser beam can be achieved. The features of the gamma-switching type surface emitting laser will be described in detail below.

The short pulse laser beam that strikes the target medium 70 is scattered and absorbed in the target medium and returns to the incident direction. The light detection module 60, which is an example of the light reception means, includes a light detector 601 (photodetector), and the light detector 601 detects a portion of the scattering light beam emitted from the target medium 70. The light detector 601 outputs a detection signal including information on a detection timing.

In the present embodiment, a single photon avalanche diode (SPAD) of a Geiger mode APD is used, but is not limited thereto. For example, an avalanche photo diode (APD) or a photomultiplier tube (PMT) can be used. Since the light beam to be detected has a weak intensity, it is preferable that the light detector has a high sensitivity and can amplify the signal. In addition, preferably, the light detectors has a high time resolution.

In terms of an optical propagation path, there are a light propagation path 70S in which the light propagates in a shallow position of 10 to 30 mm in depth of the target medium and a light propagation path 70D in which the light propagates in a deep position larger than 40 mm in depth of the target medium. In the propagation path of light, flight time (time of flight (ToF)) depends on an average optical path length, and a reception light waveform 20D passing through a deep region in the target medium has a long time delay.

In the present embodiment, a detector (e.g., SPAD) that can measure a single photon repeatedly measures an incident timing of a photon, a profile plotting a relation between the incident timing and detection frequency represents a reception light waveform. The time width of the reception light waveform 20 is around 1 to 10 nsec. An inclination portion 21 that exists in a later part of the reception light waveform 20 includes scattering light via the light propagation path 70D in which the light propagates in a deep position. The inclination portion 21 is analyzed, and an absorption coefficient μa of the light propagation path 70D of the deep region and an average optical path length indicating the depth thereof are calculated from the time course and the inclination angle. Specifically, a fitting is performed to a light diffusion equation based on the time course from the light projection to the light reception, the amount of light signal reduction, and a distance between the light projection and the light reception. Herein, the absorption coefficient μa indicates the degree of incident light reduction and is calculated as an absolute value in unit of mm⁻¹. Since the absorption coefficient μa can be calculated as an absolute value, a change of a same person from time to time or a difference between individuals can be estimated.

In FIG. 1, the light irradiation position and the light reception position are adjacent to each other in the arrangement (i.e., an adjacent arrangement), but the arrangement is not limited thereto. For example, in the case of an examination of thyroid cancer, the light irradiation position and the light reception position are arranged so as to sandwich the neck (i.e., an opposed arrangement), and an optical system is typically arranged so as to include transmission light. In addition, the opposed arrangement is more suitable for the examination of compartment syndrome of the forearm. In such cases as well, the shallow portion and the deep portion described above can be separated by the time course, and portions having different light propagation paths can be separately detected.

Each of the light source module 50 and the light detection module 60 has an aperture to prevent stray light. Preferably, these arrangements are closer to each other and may be adjacent to each other.

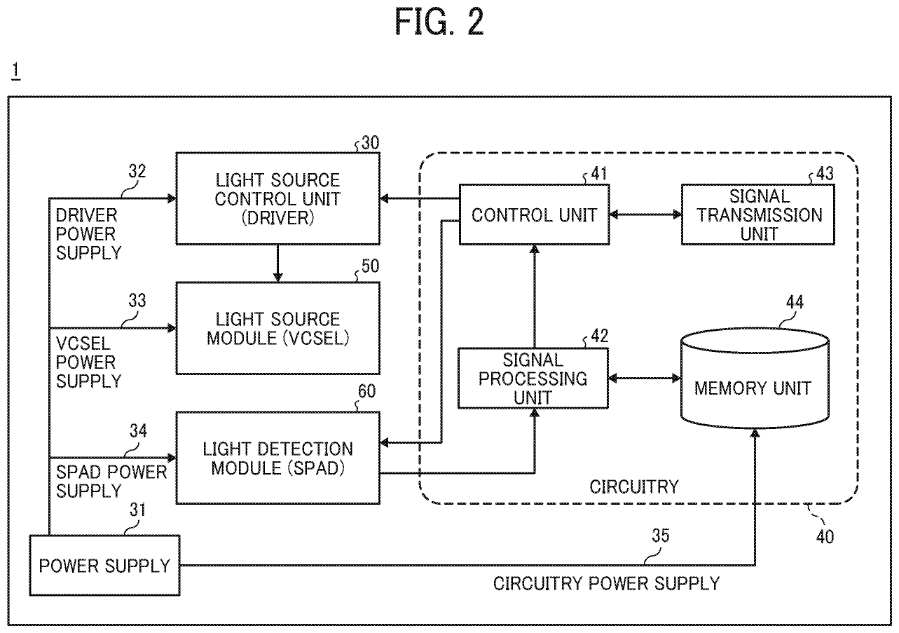

FIG. 2 is a block diagram of the TD-NIRS system 1 according to the present embodiment. The TD-NIRS system 1 includes a light source control unit 30, one or more of light source modules 50, one or more of light detection modules 60, and circuitry 40.

The TD-NIRS system 1 is, for example, an apparatus to quantitatively measure an amount of brain blood flow and have a configuration to irradiate a subject (e.g., a head) with a short pulse laser beam output from the light source module 50 and a configuration to detect the short pulse laser beam propagating inside the subject with the light detection module 60.

The circuitry 40 of the TD-NIRS system 1 includes a control unit 41, a signal processing unit 42, a memory unit 44, and a signal transmission unit 43. The power supply 31 is, for example, a battery or an external power supply, and supplies a driver power supply 32 for driving the light source control unit 30, a VCSEL power supply 33 for driving the VCSEL of the light source module 50, a light detection power supply (SPAD power supply 34) for driving the light detection module 60, and a circuitry power supply 35 for driving the circuitry 40.

The control unit 41 controls the overall operation of the TD-NIRS system 1. For example, the control unit 41 controls the operation timing of the light source module 50 and the light detection module 60, acquires data, transfers the acquired data to the signal processing unit 42 and the signal transmission unit 43, and generates and outputs processing instructions.

The light source control unit 30 is a driver of a light source, receives a trigger signal from the control unit 41, and applies current to the surface emitting laser (i.e., VCSEL 52) of the light source module 50 to emit light. The light source device includes the light source control unit 30 and the surface emitting laser (i.e., VCSEL 52). A detection signal from the light detector 601 is input to the signal processing unit 42. Further, the signal processing unit 42 compares the measurement result based on the detection signal from the light detector 601 with the result of the prior calculation stored in the memory unit 44 to calculate or estimate the optical physical value of the measurement subject.

The memory unit 44 stores the relation between the optical physical value and the light intensity ratio calculated in advance. The signal transmission unit 43 transfers the result to an external information processing device such as a smartphone or a personal computer (PC). The signal transmission unit 43 also receives a command. The operations of the signal processing unit 42, the control unit 41, the signal transmission unit 43 and the memory unit 44 are implemented by, for example, a processor. Further, the signal transmission unit 43 may send the data acquired by the detector to the signal processing unit 42 of an external information processing device to perform processing such as estimation of optical physical value. Further, as an external information processing device, in addition to using a smartphone or a PC, processing may be performed on cloud computing and the result may be sent to the smartphone or the PC.

The memory unit 44 is implemented by a memory and may include a memory built in the processor or an external memory.

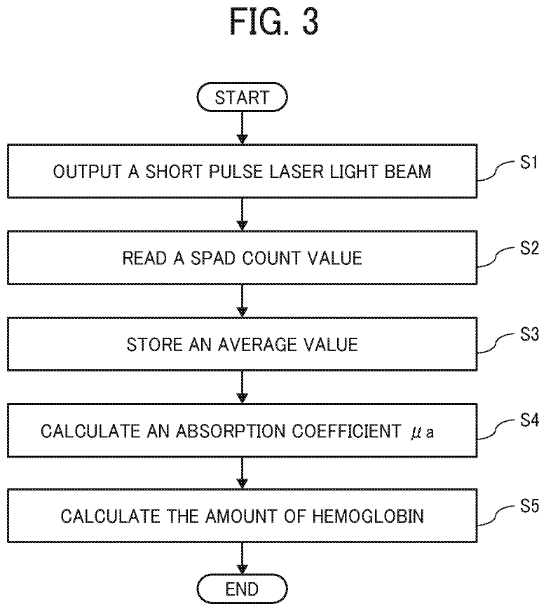

FIG. 3 is a flowchart of the processing flow of the TD-NIRS system 1. The light source module 50 outputs a short pulse laser beam and irradiates the subject with the short pulse laser beam (S1). The short pulse laser light beam propagates inside the subject and is detected by the light detection module 60.

For light emission (short pulse laser light emission), the current value is determined so that the peak intensity becomes about 100 mW. Detection values of all SPAD are read (S2) and averaged (average value) during the measurement period in consideration of the delay time of propagation in the subject.

The average value are stored in a recording medium (S3). The signal processing unit 42 calculates an absorption coefficient μa from the signal waveform (S4). Since the amount of hemoglobin can be calculated from μa (S5), the degree of oxygen saturation can be measured from the ratio of oxyhemoglobin and deoxyhemoglobin, and the amount of brain blood flow can be measured from the time course change of the amount of hemoglobin. When the measurement is carried out continuously for several minutes while a task is given to the subject, the active part of the human brain can be detected as a change in the absorption coefficient. Accordingly, the brain response to the task can be used to determine the nature of the subject.

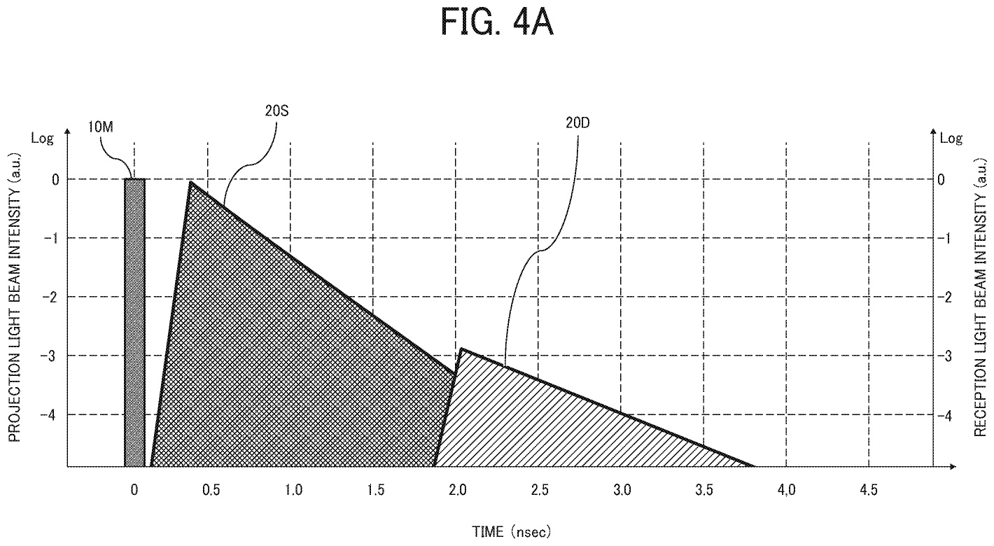

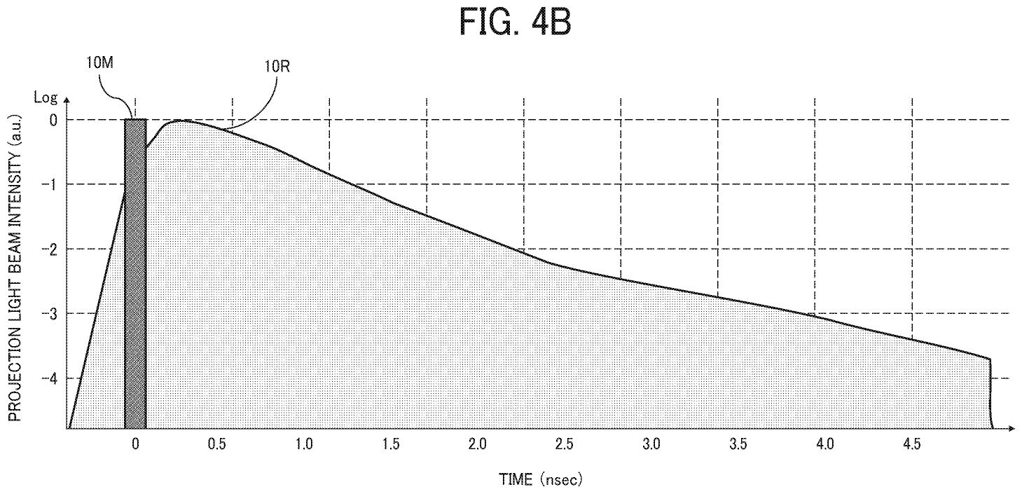

The relation between the projection light waveform 10 and the reception light waveform 20 and the relation between the light propagation paths 70S and 70D thereof will be described below. Similar to the reception light waveform 20 described above, the reception light waveforms 20S and 20D are profiles of the relation between the incident timing and the detection frequency. In FIG. 4A, a projection light waveform 10M (ideal waveform) is represented by a rectangle and an ideal model of a short pulse. The projection light waveform (ideal waveform) is a short pulse shape and does not have skirts. Specifically, in the rectangle illustrated in FIG. 4A, the pulse width is one nsec or less, and a portion of the skirts is 1/1,000 or less of the peak in the waveform. Details of the projection light waveform of the present embodiment will be described below.

In some embodiments, in the measurement system, the detector repeatedly detects an incident timing of a single photon, and the circuitry generates a profile of an incident timing and a detection frequency based on the detection signal.

The reception light waveforms 20S and 20D illustrated in FIG. 4A will be described. Herein, an example of the target medium that imitates a living body such as a human head will be described. As a converted scattering coefficient or an absorption coefficient of the standard medium, typical numerical values of a medium of a human body are used. The light emission part (e.g., a window 54 described below) of the light source module 50 and the light incidence part (e.g., an opening of a contact member 64 described below) of the light detection module 60 are arranged adjacent to each other with respect to the standard medium, so that the standard medium is a large scattering absorber having a depth of several hundred mm or more from the surface. However, the medium is not limited thereto, and as long as it is a scattering body or a transparent body, the following effects are similarly exhibited.

In FIG. 4A, the reception light waveform detected by the light detection module 60 is illustrated separately as a shallow region signal (i.e., the reception light waveform 20S) from a shallow region and a deep region signal (i.e., the reception light waveform 20D) from a deep region. Although the reception light waveform 20S (i.e., the shallow region signal) depends on the distance between the light emission part of the light source module 50 and the light incident parts of the light detection module 60, in a case where the distance is closer according to the present embodiment, the waveform reaches the peak in several hundred psec after projection of the projection light waveform 10M. After the peak, the detection intensity decreases exponentially, and after about 2 nsec, the light intensity decreases by nearly three orders of magnitude.

By contrast, the scattering light passing through a deep position such as 40 mm has a time delay of about 2 nsec. In the reception light waveform 20D (i.e., the deep region signal) illustrated in FIG. 4A, the intensity of the reception light is detected to be about three orders of magnitude lower than that of the signal from a shallow position. In the case of the ideal waveform as illustrated in FIG. 4A, the reception light waveform 20D (i.e., the deep region signal) from the deep region is slightly larger than the reception light waveform 20S (i.e., the shallow region signal) from the shallow region after about 2 nsec from the light projection.

Thus, in the case of the ideal waveform, the reception light waveform 20D (deep region signal) in a deep region can be detected. Further, at this time, since the time course of the reception light waveform 20S (i.e., the shallow region signal) and the reception light waveform 20D (i.e., the deep region signal) differs by about 2 nsec, the signals can be detected independently by time-resolved detection with high accuracy. The activation of brain activity at a deep position can be accurately detected without being affected by the presence or absence of activation of brain activity at a shallow position.

FIG. 4B is a diagram of the projection light waveform 10R as a comparative example. The projection light waveform (comparative example) has skirts. The skirts indicate a state in which time course of reduction of the light emission intensity is longer, and the light emission lasts longer as compared with the pulse width. In a typical use situation of a laser such as an optical communication or a laser projector, if the light intensity is reduced to about ⅕, and there is the skirts, the skirts does not affect the use situation. However, in the case of the present embodiment, even if the intensity of the skirts is about 1/1,000 of the peak intensity, the signal detection may cause difficulties in detection in present embodiment described later. Even if a short pulse light having a full width at half maximum (FWHM) of about several hundred picoseconds (psec), a long projection light waveform of the 1/e² pulse width or time course of the “skirts” in which the intensity reduces to 1/1,000 of the peak intensity exceeds several nsec may have a difficulty described later.

An issue to be addressed on the skirts of the projection waveform will be described below. In the projection light waveform 10R (comparative example), the FWHM is about 500 psec. Further, the time width in which the light intensity becomes 1/10 of the peak intensity is about 1.1 nsec, 1/100 is about 2.1 nsec, and 1/1,000 is about 3.6 nsec.

The projection light waveform 10R (comparative example) is a typical gain-switching type laser light beam in which a semiconductor laser is driven by a transistor of a GaN substrate.

FIG. 4C is a diagram of the reception light waveform with respect to the projected light waveform (comparative example). The reception light waveform 20D (signal from the deep portion) is not greatly affected by the projection light waveform and does not substantially change as illustrated in the drawing. By contrast, the waveform of the reception light waveform 20S (signal from a shallow portion) is greatly changed. In FIG. 4A, a portion of about 2 nsec in which the intensity is decreased by about three orders of magnitude from the peak intensity. However, in FIG. 4, the portion of about 2 nsec is significantly affected.

The reception light waveform 20S (signal from a shallow portion) in FIG. 4C by the projection light waveform 10R (comparative example) in FIG. 4B is reduced by only about two orders of magnitude in 2 nsec. Thus, the intensity of 2 nsec of the projection light waveform 10R (comparative example) is also reduced by about two orders of magnitude with respect to the peak intensity.

In contrast to the reception light waveform 20S (signal from a shallow portion), the reception light waveform 20D (signal from a deep portion) is small for any time period, and the signal is buried. In such a state, the detection of the reception light waveform 20D (signal from a deep portion) has a difficulty in its accuracy.

For the reason described above, in order to obtain a signal from a deep portion, as illustrated in FIG. 4A, it is preferable that there is no “skirts” in the projection light waveform.

Since the projection light is put into the human body, the amount of the light is limited. In order to obtain a signal from a deeper region, the effect of the projection light illustrated in FIG. 4A is excluded.

Degree of reduction of the peak of the projection light has a larger influence on information obtained as a NRS signal. Although a signal by light passing through a deep portion in the skirts of the projection light waveform can be used, the signal is not preferable to use because of low accuracy.

From the examination described above, the present inventors have found that a time width at an intensity lower than the FWHM characterizes a short pulse light source. Specifically, a light source having a time width of 200 psec or less, in which the light intensity becomes 1/e² of the peak intensity, is preferable. Further, a light source that outputs a pulse light beam whose light intensity is attenuated to 1/100 or less at 1 nsec from the light emission peak is preferable.

In some embodiments, in the light source device, the surface emitting laser emits the light having a light intensity of 1/100 or less with respect to the peak after one nanosecond of the peak.

An application example in which a large effect is obtained when the light intensity at one nsec (1 nanosecond) from the emission peak becomes 1/100 or less will be described below. When measuring the brain, skin blood flow in the head may cause difficulties. The skin blood flow is the blood flow existing in the scalp. When the brain is measured, the blood flow causes artifact to include a measurement error. The depth of skin blood flow is 1 mm, and the depth of brain blood flow is about 20 mm because the brain flood flow exists in a deep region beyond the thickness of skull and cerebrospinal fluid.

Assuming that the light source module 50 and the light detection module 60 are arranged at positions as close as possible, when the difference between 1 mm and 20 mm in the depth corresponds to a difference between 2 mm and 40 mm in the length of the propagation path as a reciprocal length.

As a result, positions indicated by the skin blood flow and the brain blood flow are different in the reception light waveform. The propagation path of skin blood flow is about 2 mm, and the position is several hundred psec. By contrast, the propagation path of the cerebral blood flow is 40 mm, and the position is about 1 nsec. In the light projection waveform, the light intensity at 1 nsec from the light emission peak is sufficiently reduced. Specifically, when the light intensity at 1 nsec from the light emission peak is reduced to 1/100 even if there is an effect of the skin blood flow, the change of the brain blood flow becomes dominant, so that the adverse effect of the skin blood flow is limited. Thus, the skin blood flow and the brain blood flow can be accurately separated.

By contrast, when the skirts from the light emission peak is more than 1 ns, the change of the skin blood flow is reflected in the position in which the brain blood flow is reflected, and the skin blood flow and the brain blood flow are not distinguished from each other.

An example of enabling discrimination at a further position will be described. For the determination at the deep position, the intensity of the projection light waveform becomes 1/1,000 or less to the emission peak at 2 nsec from the light projection. As a specific example, an application for determining the activation state for each part of the brain will be described below. In terms of the brain blood flow, there are a deep position and a shallow position in the region of the brain. For example, as the deep position, the cingulate gyrus is located at a position of about 40 mm. By contrast, the frontal cortex such as the dorsolateral prefrontal cortex (DLPFC) is located at a position about 20 mm. By detecting these two positions simultaneously and independently, the brain function can be understood from various aspects.

The light propagation path can be simulated by measuring the optical constants of brain tissue. The time course in which the light beam passing through a deep position of about 40 mm such as the cingulate gyrus returns is about 2 nsec. When a portion of the skirts of the light in the projection light waveform passing through the shallow position exists in this time region, the detected light may not be determined whether the detected light comes from the shallow portion or the deep position. Thus, the amount of the light propagating through the shallow position and returning at the time course about 2 nsec is reduced as much as possible.

In a scattering body such as a living body, the light intensity from the deep position around 40 mm is about 1/1,000 of the light intensity from the shallow position. In order to distinguish the deep position and the shallow position, the light intensity of the skirt of the light source, which is after 2 nsec, is reduced to 1/1,000. As a result, the signal at the deep position becomes stronger than the signal at the shallow position, and the signal at the deep position can be accurately detected.

In some embodiments, in the measurement system, the surface emitting laser emits the pulse light beams having a light intensity of 1/100 or less with respect to the peak after one nanosecond after the peak.

Further, the light source is a light source to irradiate the subject of a scattering body or a transparent body with a laser light beam, and, preferably, a surface emitting laser having a 1/e² pulse width of 200 psec or less that is a time width of 1/e² of the peak of the light intensity.

In the present embodiment, the 1/e² pulse width is 200 psec, and if the 1/e² pulse width is simply divided by 2, the peak falling becomes 100 psec. After about 100 psec from the peak, since the light intensity in the projection light waveform is reduced to 1/e², a distance of several mm as an optical path length can be separated. As a structure of the skin, the layer referred to as the dermis layer is about 2 mm in thickness, and there are subcutaneous fat and muscle layer under the dermal layer. The dermis layer can be separated from the subcutaneous fat and the muscle layer by the light source according to the present embodiment.

Further, since the skin layer on the surface has a thickness of several hundred micrometers, and only the skin layer can be recognized by visual observation, there is also an advantage by separating information of the dermis layer under the skin layer. Since it takes about 100 psec to decrease the light intensity to 1/e² of the peak intensity, a time region before 100 psec at which the effect of the skin surface appears and a time region after 200 psec at which the effect of the dermis layer appears can be separated. As a result, a blood flow state of the dermis layer that is not visibly recognized can be independently quantified, and a comprehensive judgment for the skin can be made.

In some embodiments, a measurement system includes: the light source device according to the sixteenth aspect; a detector to detect multiple scattering light beams propagating inside the subject and output a detection signal including information of a detection timing, the circuitry to: input and process the detection signal; and control the surface emitting laser to emit pulse light beams to an interior of the subject multiple times, and the pulse light beam having a time width, a light intensity of which is 1/e² with respect to the peak, of 200 picoseconds or less.

A gamma-switching type surface emitting laser as a light source used in the present embodiment will be described. The concept of the gamma-switching type surface emitting laser and the projection light waveform of the present embodiment will be described below.

As a means for achieving a short pulse of 1 nsec or less, for example, gain switching, Q-switching, or mode locking is used. Gain switching is a means for achieving a pulse width of 100 psec or less in FWHM by utilizing a relaxation oscillation phenomenon. Since gain switching can be achieved only by controlling the pulse current, the configuration is simpler than the configuration of Q-switching or mode locking.

However, in gain-switching, since the relaxation oscillation phenomenon is used, multiple pulse trains are likely to output after the first pulse, or pulse-tail (skirts) whose attenuation is slower is likely to output after the relaxation oscillation is finished.

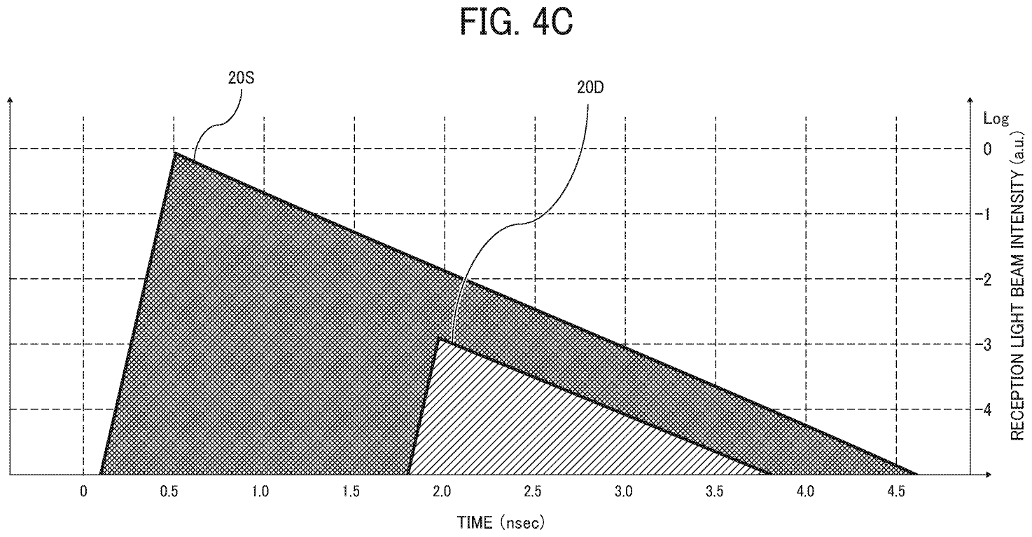

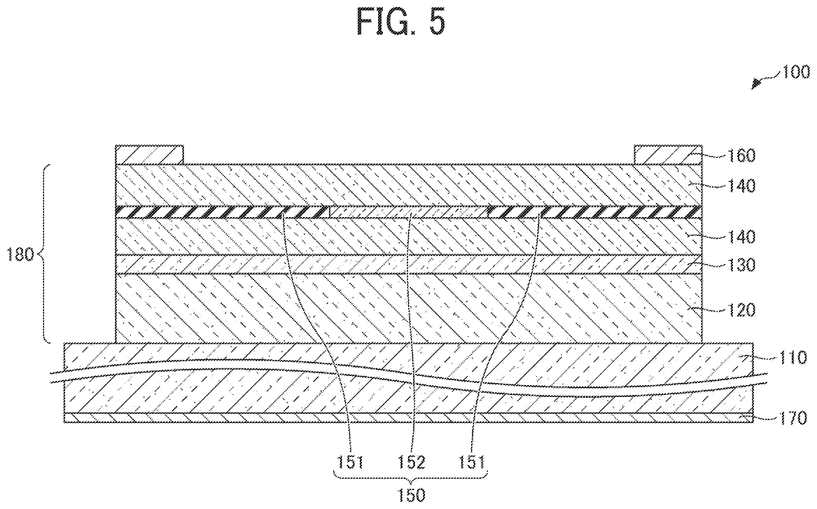

The structure according to the present embodiment is illustrated in FIG. 5. The surface emitting laser 100 according to the present embodiment is, for example, a vertical cavity surface emitting laser (VCSEL) employing oxidation confinement. A surface emitting laser 100 includes an n-type GaAs substrate 110, an n-type distributed Bragg reflector (DBR) 120, an active layer 130, a p-type DBR 140, an oxidation confinement layer 150, an upper electrode 160, and a lower electrode 170.

In the present embodiment, a light beam is emitted in a direction perpendicular to the surface of the n-type GaAs substrate 110. Herein, a direction perpendicular to the surface of the n-type GaAs substrate 110 may be referred to as a longitudinal direction, and a direction parallel to the surface of the n-type GaAs substrate 110 may be referred to as a lateral direction or in-plane direction.

The n-type DBR 120 is on the n-type GaAs substrate 110. The n-type DBR 120 is, for example, a semiconductor multilayer reflection mirror formed by laminating multiple n-type semiconductor films. The active layer 130 is on the n-type DBR 120. The active layer 130 includes, for example, multiple quantum well layers and a barrier layer. The resonator includes the active layer 130. The p-type DBR 140 is on the active layer 130. The p-type DBR 140 is, for example, a semiconductor multilayer reflection mirror formed by laminating multiple p-type semiconductor films. In the resonator, the active layer 130 is disposed at a position closer to an antinode rather than a middle position between the antinode and the node of the standing wave of the oscillation light beam. When the active layer 130 is disposed at a position corresponding to the antinode of the standing wave, the luminous efficiency becomes maximum.

The upper electrode 160 is in contacts with the top surface of the p-type DBR 140. The lower electrode 170 is in contact with the bottom surface of the n-type GaAs substrate 110. A pair of the upper electrode 160 and the lower electrode 170 is an example of an electrode pair. However, the position of the electrode is not limited thereto, and may be a position in which current can be injected into the active layer. For example, an intracavity structure in which the electrodes are disposed directly on the spacer layer of the resonator instead of via the DBR may be used.

The p-type DBR 140 includes, for example, an oxidation confinement layer 150. The oxidation confinement layer 150 includes Al. The oxidation confinement layer 150 includes an oxidized region 151 and a non-oxidation region 152 in a plane perpendicular to the light emission direction. The oxidation region 151 has an annular planar shape and surrounds the non-oxidation region 152. The non-oxidized region 152 includes a p-type AlAs layer 155 and two p-type Al0.85Ga0.15As layers 156 sandwiching the p-type AlAs layer 155 in the longitudinal direction. The oxidized region 151 includes AlOx. The refractive index of the oxidized region 151 is lower than that of the non-oxidation region 152. For example, the oxidation region 151 has a refractive index of 1.65, the p-type AlAs layer 155 has a refractive index of 2.96, and the p-type Al0.85Ga0.15As layer 156 has a refractive index of 3.04.

In plan view, a portion inside the inner edge of the oxidized region 151 of the mesa 180 is an example of a high refractive index region, and a portion outside the inner edge of the oxidized region 151 of the mesa 180 is an example of a low refractive index region. A p-type AlxGal-xAs layer (0.70≤x≤0.90) may be provided instead of the p-type Al0.85Ga0.15As layer 156. In the present embodiment, the mesa 180 includes the p-type DBR 140, the active layer 130, and the n-type DBR 120. However, in the present embodiment in which the current confinement region is formed by the oxidation confinement, at least the oxidation confinement layer 150 and the semiconductor layer disposed above the oxidation confinement layer 150 may be formed in a mesa shape. Further, by forming the active layer so that the mesa includes at least the active layer, light generated in the active layer is prevented from leaking in the lateral direction.

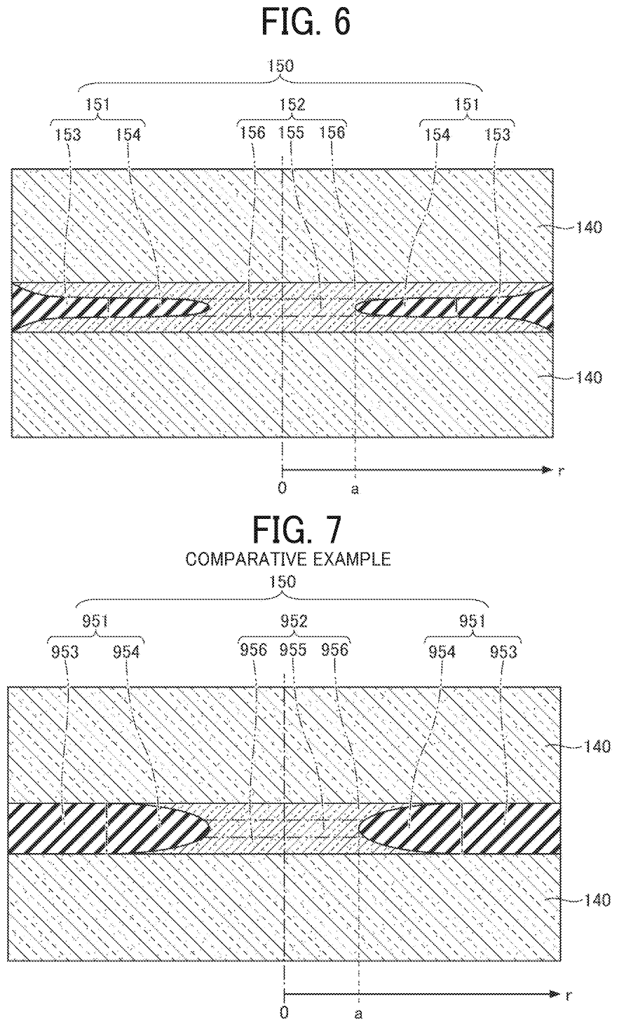

The oxidation confinement layer 150 will be described in detail. FIG. 6 is a cross-sectional view of the oxidation confinement layer and the surroundings according to the present embodiment.

As illustrated in FIG. 6, the oxidation region 151 has an outer region 153 having an annular shape and an inner region 154 having an annular shape in plan view. The outer region 153 having an annular shape is exposed on the side surface of the mesa 180. The outer region 153 having an annular shape is a region in which the thickness changes so that the contact surface of the surface is positioned outside the oxidized region 151 in a cross-sectional view, and the inner region 154 is a region in which the thickness changes so that the contact surface of the surface is positioned inside the oxidized region 151 in a cross-sectional view. The inner region 154 is inside the outer region 153. The thickness of the inner region 154 coincides with the thickness of the outer region 153 at the boundary with the outer region 153 and becomes thinner as approaching the center of the mesa 180.

The inner region 154 has a taper shape in which the thickness gradually increases from the inner edge to the boundary with the outer region 153 in cross-sectional view. The non-oxidation region 152 is inside the outer region 153. A portion of the non-oxidation region 152 sandwiches the inner region 154 in the longitudinal direction. Another portion of the non-oxidation region 152 is inside the inner edge of the inner region 154 in plan view. For example, the thickness of the non-oxidation region 152 is 35 nm or less.

In some embodiments, in the light source device, the surface emitting laser includes: a first refractive region having a first refractive index; and a second refractive region surrounding the first refractive region and having a second refractive index lower than the first refractive index of the first refractive region, the second refractive region includes an oxide confinement, the first refractive region has a thickness of 35 nm or less, and the second refractive region has a thickness twice or less of the thickness of the first refractive region at a position three micrometers from a tip of a boundary between the second refractive region and the first refractive region.

The outer region 153 may have a thickness larger than the thickness of the non-oxidation region 152. In the present embodiment, the thickness of the non-oxidation region 152 is the thickness of a portion in the vicinity of the center of the mesa 180 rather than the thickness of the inner edge of the oxidized region 151 (inner edge of the inner region 154). For example, the distance from the side surface of the mesa 180 to the inner edge of the oxidized region 151 is in the range of about 8 μm to 11 μm.

The oxidized region 151 is formed by, for example, oxidation confinement of a p-type AlAs layer and a p-type Al0.85Ga0.15As layer. For example, the oxidized region 151 is formed by oxidation of the p-type AlAs layer and the p-type Al0.85Ga0.15As layer under a condition of water vapor with higher temperature. Even if the same p-type AlAs layer and p-type Al0.85Ga0.15As layer are oxidized, the structures of the oxidation confinement layer obtained from the p-type AlAs layer and the p-type Al0.85Ga0.15As layer may be different depending on the oxidation conditions.

Thus, even if the structures before oxidation of, for example, the p-type AlAs layer and the p-type Al0.85Ga0.15As layer, which are layers becoming the oxidation confinement layer 150 by oxidation, are the same, the oxidation confinement layer 150 including the oxidized region 151 and non-oxidation region 152 may not be obtained.

The operation and effect of the present embodiment will be described in comparison with a comparative example. FIG. 7 is a cross-sectional view of the oxidation confinement layer and the surroundings according to the present embodiment.

In the comparative example, the oxidation confinement layer 150 has an oxidation region 951 and a non-oxidation region 952 instead of the oxidized region 151 and the non-oxidation region 152. The oxidation region 951 has an annular planar shape and surrounds the non-oxidation region 952. The non-oxidation region 952 includes a p-type AlAs layer 955 and two p-type Al0.85Ga0.15As layers 956 sandwiching the p-type AlAs layer 955 in the longitudinal direction. The oxidation region 951 includes AlOx.

The oxidation region 951 has an outer region 953 having a annular shape and an inner region 954 having a annular shape in plan view. The outer region 953 having a annular shape is exposed on the side surface of the mesa 180. The thickness of the outer region 953 is constant in the in-plane direction. The inner region 954 is inside the outer region 953. The thickness of the inner region 954 coincides with the thickness of the outer region 953 at the boundary with the outer region 953, and becomes thinner closer to the center of the mesa 180.

The inner region 954 has a tapered shape in which the thickness gradually increases from the inner edge to the boundary with the outer region 953 in a cross-sectional view. The non-oxidation region 952 is inside the outer region 953. A portion of the non-oxidation region 952 sandwiches the inner region 954 in the longitudinal direction. Another portion of the non-oxidation region 952 is inside the inner edge of the inner region 954 in plan view. For example, the distance from the side surface of the mesa 180 to the inner edge of the oxidation region 951 is in the range of about 8 μm to 11 μm. The thickness of the oxidation region 951 and the thickness of the non-oxidation region 952 are equal to the thickness of the oxidation confinement layer 150.

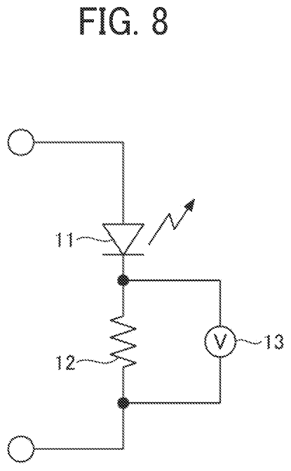

Actual measurement results of the present embodiment and the comparative example will be described. FIG. 8 is an equivalent circuit diagram of a circuit used for the actual measurement.

In the circuit, a resistor 12 for current monitoring is connected in series to a surface emitting laser 11 corresponding to the first embodiment or the comparative example. A voltmeter 13 is connected in parallel to the resistor 12. The light beam output from the surface emitting laser 11 was received by a broadband high-speed photodiode and converted into a voltage signal, and the voltage signal was observed by an oscilloscope.

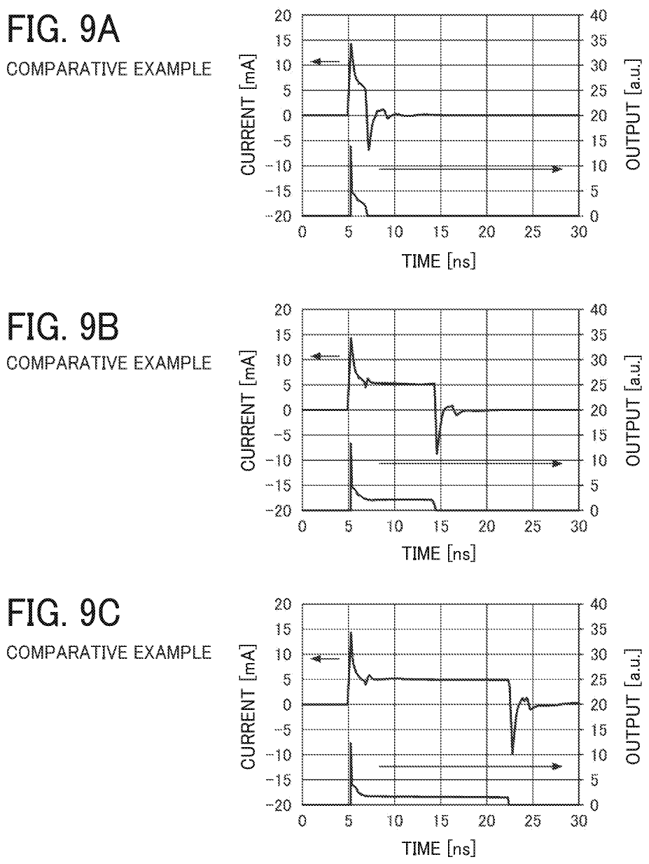

FIGS. 9A to 9C are graphs of actual measurement results as the comparative example. FIG. 9A is a graph of the result of actual measurement in a case of a width of pulse current of about 2 ns. FIG. 9B is a graph of the result of actual measurement in a case of the width of pulse current of about 9 nsec. FIG. 9C is a graph of the result of actual measurement in a case of the width of pulse current of about 17 nsec. In the actual measurements of FIGS. 9A to 9C, the magnitude of the bias current and the amplitude of the pulse current are common. FIGS. 9A to 9C illustrates the current flowing through the resistor 12 and the light output measured by the high-speed photodiode. The current flowing through the resistor 12 can be calculated using the voltmeter 13.

As illustrated in FIGS. 9A to 9C, in the comparative example, the optical pulse is output immediately after the pulse current injection regardless of the width of the pulse current, the equilibrium state is maintained after the optical pulse output until the pulse current injection is stopped, and constant pulse-tail is output. The first optical pulse is due to relaxation oscillation and is a typical gain-switching drive. Even if the pulse width of the current is changed, the timing at which the optical pulse is generated does not change. The optical pulse generated by the relaxation oscillation is generated immediately after the carrier density in the laser resonator exceeds the threshold carrier density.

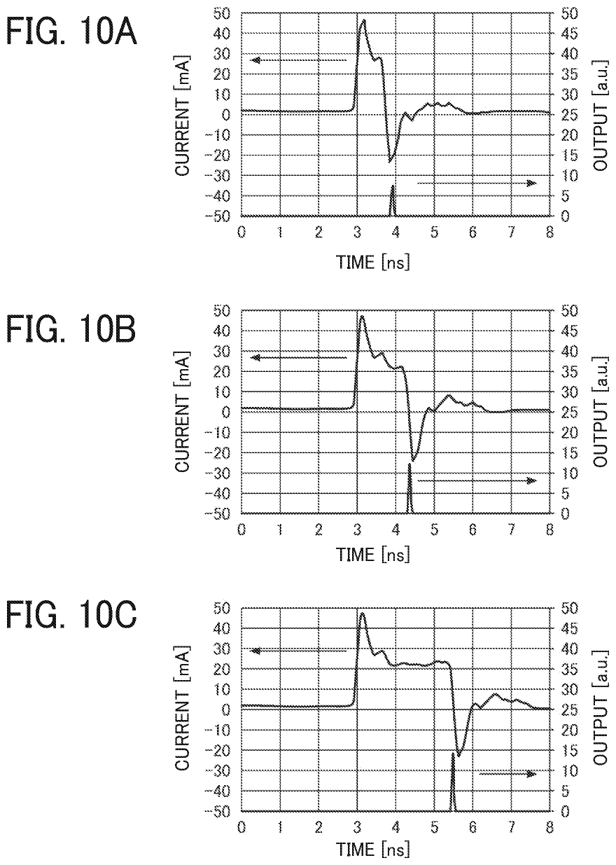

FIGS. 10A to 10C are graphs of actual measurement results according to the present embodiment. FIG. 10A is a graph of the result of actual measurement in a case of a width of pulse current of about 0.8 nsec. FIG. 10B is a graph of the result of actual measurement in a case of the width of pulse current of about 1.3 nsec. FIG. 10C is a graph of the result of actual measurement in a case of the width of pulse current of about 2.5 nsec. In the actual measurements of FIGS. 10A to 10C, the magnitude of the bias current and the amplitude of the pulse current are common. FIGS. 10A to 10C illustrates the current flowing through the resistor 12 and the light output measured by the high-speed photodiode. The current flowing through the resistor 12 can be calculated using the voltmeter 13.

As illustrated in FIGS. 10A to 10C, in the present embodiment, the optical output does not occur in the state in which the pulse current is injected, and the optical pulse is output immediately after the pulse current injection is reduced. Moreover, the pulse-tail after the optical pulse output almost vanishes. If the optical output is due to a gain-switching and a width of the pulse current is changed, the timing at which the optical pulse is generated does not change. By contrast, in the present embodiment, the optical pulse is output triggered by a decrease in the pulse current injection. Thus, the optical output in the present embodiment is not typical gain-switching using the relaxation oscillation phenomenon.

As described above, the first embodiment is clearly different from the comparative example in the mechanism and aspect of optical output. This difference is explained as follows.

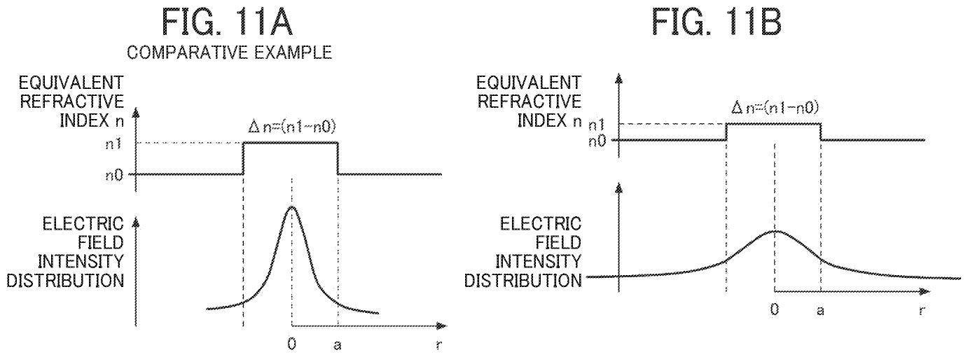

In the surface emitting laser, the laser light beam propagates in the resonator in a direction perpendicular to the oxidation confined layer. Thus, the thicker the oxidation confinement layer is, the longer the equivalent waveguide length depending on the refractive index difference is. As a result, the lateral optical confinement effect becomes larger. When the DBR including the oxidation confinement layer is regarded as an equivalent waveguide structure, and the equivalent refractive index difference is larger as illustrated in FIG. 11A, the electric field intensity distribution of the laser light beam is collected around the center. By contrast, the equivalent refractive index is small as illustrated in FIG. 111B, the distribution of electric field intensity spreads to the region around the oxidation region.

When the present embodiment is compared with the comparative example, in the first embodiment, since the oxidation confinement layer 150 includes the inner region 154, the equivalent refractive index becomes small in the present embodiment. Accordingly, in the comparative example, as illustrated in FIG. 11A, the electric field intensity distribution of the laser light is collected around the center. By contrast, in the first embodiment, as illustrated in FIG. 11B, the electric field intensity distribution of the laser light expands to the oxidized region 151.

Herein, an optical confinement coefficient in the lateral direction is defined as a ratio of “integrated intensity of the electric field in the same radius region as the current passage region” to “integrated intensity of the electric field in the lateral cross section passing through the center of the surface emitting laser element”, and an expression (1) is defined below.

$${Γ}_{r} = \frac{{\int }_{0}^{a} {\int }_{0}^{2 π} {\vert E \vert}^{2} \cdot rd Φ dr}{{\int }_{0}^{∞} {\int }_{0}^{2 π} {\vert E \vert}^{2} \cdot rd Φ dr} \qquad ( 1 )$$

where a corresponds to a radius of a region through which current passing, Φ is represents a rotation direction in which a rotational axis is perpendicular to the substrate.

A model of a phenomenon that occurs when the injection of the pulse current is stopped will be described. In a state in which the pulse current is injected, the current path is concentrated around the center of the mesa due to the oxidation confinement layer, and the carrier density is higher. At this time, in the non-oxidation region having a higher carrier density, the effect of reducing the refractive index due to the carrier plasma effect occurs. The carrier plasma effect is a phenomenon in which the refractive index decreases in proportion to the free carrier density. For example, in the NPL 1, the amount of change in the refractive index is expressed by an expression (2) below.

$$Δ n \approx - 4 \times {10}^{- 21} [ {cm}^{3} ] \times N [ 1 / {cm}^{3} ] \qquad ( 2 )$$

where N is a carrier density.

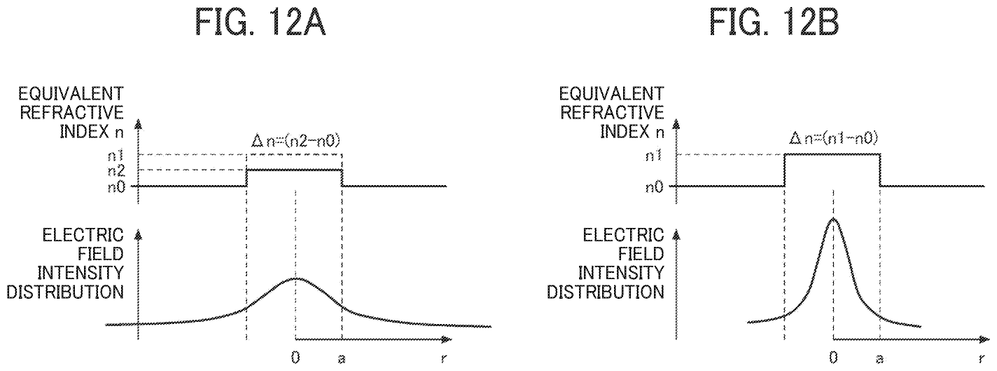

FIG. 12A is a diagram of the equivalent refractive index and the electric field intensity distribution during a period in which the pulse current is injected, and FIG. 12B is a diagram of the equivalent refractive index and the electric field intensity distribution during a period in which the pulse current is stopped and decreases. During the period in which the pulse current is injected, the carrier plasma effect works in a direction to cancel the equivalent refractive index difference (n1−n0) caused by the oxidation confinement layer, and the equivalent refractive index difference becomes (n2−n0). When the injection of the pulse current decreases in such a state, the action of the carrier plasma effect disappears, and the equivalent refractive index difference returns to (n1−n0). As a result, photons spreading to the periphery of the mesa are collected at the center of the mesa, and the photon density in the non-oxidation region is increased.

Thus, the lateral optical confinement is changed to a stronger state. When the injection of the pulse current is stopped, the carriers stored in the resonator decrease over the carrier lifetime. However, when the lateral optical confinement becomes stronger before the carrier density completely attenuates, stimulated emission starts, the stored carriers are consumed at once, and an optical pulse is output. The period in which the pulse current is injected is an example of the current injection period, and the period in which the injection of the pulse current is stopped and the pulse current is reduced is an example of the current reduction period. In some embodiments, in the light source device, the surface emitting laser includes: an active layer; multiple reflectors sandwiching the active layer between the multiple reflectors; and an electrode pair connected to a power supply to inject current into the active layer. The circuitry generates a drive signal having: a current injection period in which current is injected into the active layer; and a current reduction period in which a value of the current injected into the active layer is reduced after the current injection period, and the surface emitting laser does not oscillate in the current injection period, and the surface emitting laser oscillates in the current reduction period.

In some embodiments, in the measurement system, the surface emitting laser includes: an active layer; multiple reflectors sandwiching the active layer between the multiple reflectors; and an electrode pair connected a power supply to inject current into the active layer. The circuitry generates a drive signal has: a current injection period in which current is injected into the active layer; and a current reduction period in which a value of the current injected into the active layer is reduced after the current injection period, and the surface emitting laser does not oscillate in the current injection period, and the surface emitting laser oscillates in the current reduction period.

In the present embodiment, a continuous optical pulse train is less likely to occur after the optical pulse output occurs. This is because the injection of the pulse current is reduced when the optical pulse is generated, and the relaxation oscillation is less likely to occur.

In addition, the pulse-tail is less likely to occur after light pulse output occurs. This is because the injection of the pulse current is reduced when the optical pulse is generated, and the carrier density is less likely to increase.

Further, since the optical pulse is output immediately after the pulse current injection is stopped, the timing at which the optical pulse is output can be controlled at any time.

Further, the optical pulse width generated in the first embodiment is shorter than the pulse current injection width. Since the pulse current width is not shortened even when the current is increased, parasitic inductance has less effect.

In the typical gain-switching, in order to reduce the output of the pulse-tail, it is conceivable to stop the current injection immediately after the output of the optical pulse. Ideally, the current pulse width has several hundred psec. However, since the current waveform is affected by the parasitic component, such a current pulse having a smaller pulse width is less likely to produce. In particular, when the magnitude of the current is as large as 10 A or more, the current injection in a time of 100 psec or less immediately after the optical pulse output is less likely to stop. A configuration of circuitry disadvantageously becomes large because a large condenser is used. However, a large current and short current pulse can be generated by using a capacitor that can store a large amount of charge and using a circuit configuration in which the stored charge flows instantaneously.

By contrast, the rise time of a light pulse in a gamma-switching type surface emitting laser becomes shorter when the threshold carrier density Nth decreases earlier than the carrier lifetime. Thus, as the lateral optical confinement coefficient Fr increases faster, the rise time becomes shorter. The decay time of the optical pulse depends on the photon lifetime. As a result, since the input current pulse width may be longer, the specification of the circuit configuration is relaxed, and application to a smaller sensor such as a wearable sensor can be achieved.

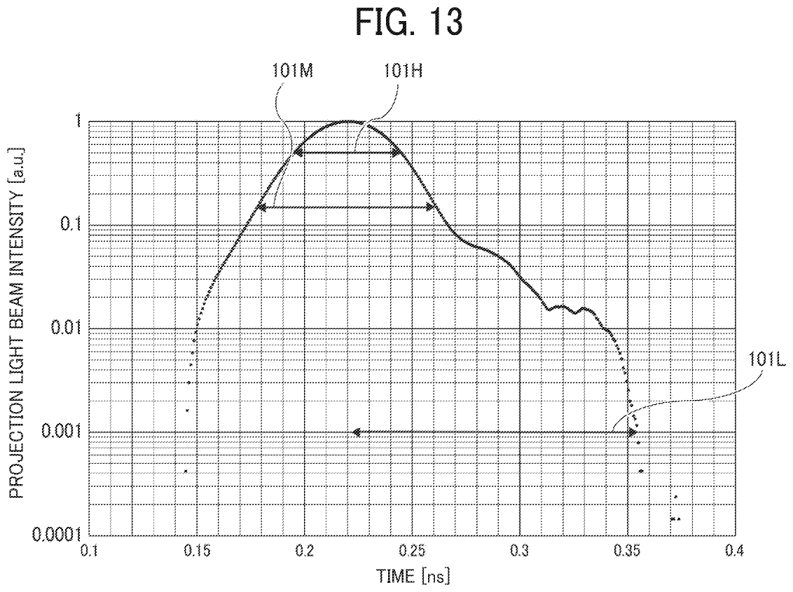

FIG. 13 is a graph of the result of the actual measurement of the optical pulse. As optical pulse widths, a full width at half maximum (FWHM) 101H and a 1/e² pulse width 101M are indicated by arrows, respectively.

The full width at half maximum (FWHM) and the 1/e² pulse width in the actual measurement result obtained were the FWHM of 40 to 60 psec and the 1/e² pulse width of 80 to 110 psec. The ratio of the 1/e² pulse width to the FWHM (i.e., (1/e² pulse width) divided by (FWHM)) was about 1.7 to 1.8. Based on the definition of the Gaussian function, since the ratio of the 1/e² pulse width to the FWHM is 1.70, the optical pulse of the present embodiment has a waveform close to the Gaussian function. By contrast, according to the actual measurement results of the comparative examples (FIGS. 9A, 9B, and 9C), since constant pulse-tail is output after the pulse light, the light intensity does not become 1/e² or less of the peak value until the current injection is stopped. Thus, since the 1/e² pulse width in the comparative example depends on the width of the pulse current, an optical pulse width in the order of picoseconds is less likely to obtain.

Further, in FIG. 13, the time course from the peak position to the position in which the light intensity becomes 1/1,000 of the peak is represented by an arrow 101L. Reading from the numerical value of the graph, it is reduced to 1/1,000 or less after about 130 psec from the peak position. As described above, since the surface emitting laser of the present embodiment does not emit light until the next pulse comes, naturally, the light is 1/1,000 or less even after 2 nsec for measurement. This shape is equivalent to the ideal optical pulse illustrated in FIG. 4A.

A reception light waveform received by the SPAD sensor will be described. A histogram can be obtained by repeating the measurement in the case where a light reception waveform is obtained by shifting by, for example, a light reception time of 40 psec per 1 by gating.

At this time, the SPAD sensor may be a sensor that repeatedly integrates the output of one pixel. However, an SPAD array having a silicon photomultiplier (SiPM) structure that integrates the count number for the large number of pixels (e.g., 1,000) and outputs has high measurement efficiency. It may also be a combination thereof. The dynamic range of the reception light waveform is obtained by multiplying the number of SPAD arrays by the number of repeated measurements.

Since the intensity of the reception light signal from the human body is four to five orders of magnitude lower than the intensity of the incident signal, the dynamic range of the reception light waveform is at least two to three orders of magnitude in order to obtain a signal in a deeper region (with a larger ToF delay). Thus, the number of SPAD arrays times the number of repeated measurements is about 10⁶ to 10⁸. Details of the above are described, for example, in NPL 2.

Since the larger the number of repeated measurements increases the measurement time, the number of SPAD arrays is increased as much as possible. On the other hand, since the lower limit value of the reception light waveform is determined by the dark count (DR) of the SPAD device, the number is determined by the dark count times the number of array, which leads to a reduction in the dynamic range.

Since there is an upper limit to the amount of incident laser light because the laser light enters the human body, an optimum combination of the dark count, the number of arrays, and the number of repeated measurements are considered. For the human skin safety, the upper limit of the laser output is determined to be, for example, a peak power of 100 to 200 mW.

The lower limit is determined by the DR to be, for example, 100 to 1,000 count per second. In order to set the DR to 2 to 3 digits, for example, the number of arrays is set to 16 to 64, and the number of repeated measurements is set to 1,024 to 65,536 times.

Further, in the present embodiment, since a short pulse laser beam without skirts can be output, the repetition frequency for the pulse light output can be set to about 10 to 100 MHz. As the repetition frequency is larger, the number of measurements per time can be increased, and the measurement time can be reduced. On the other hand, the upper limit of the average output power is limited in consideration of safety as described above. The average output power is calculated as the peak power of the laser output times a pulse width divided by the repetition period. The average output power is about 200 W or less as a typical reference when the human skin is used as a subject. For example, when the peak power is 100 mW, the pulse width is 100 psec, and the repetition period is 100 nsec (i.e., frequency is 10 MHz), the average output power is 100 μW. For example, when the peak power is 20 mW, the pulse width is 100 psec, and the repetition period is 10 nsec (i.e., frequency is 100 MHz), the average output power is 200 μW. When the pulse light having a smaller peak power is used, the probability of detection of a signal is lower than the probability when the pulse light having a large peak power is used, but the number of signal counts can be increased by increasing the repetition frequency (e.g., 100 MHz). By contrast, the number of the signal count can be increased by increasing the probability of detecting a signal by setting the peak power to a value close to the upper limit instead of reducing the repetition period (e.g., 10 to 20 MHz).

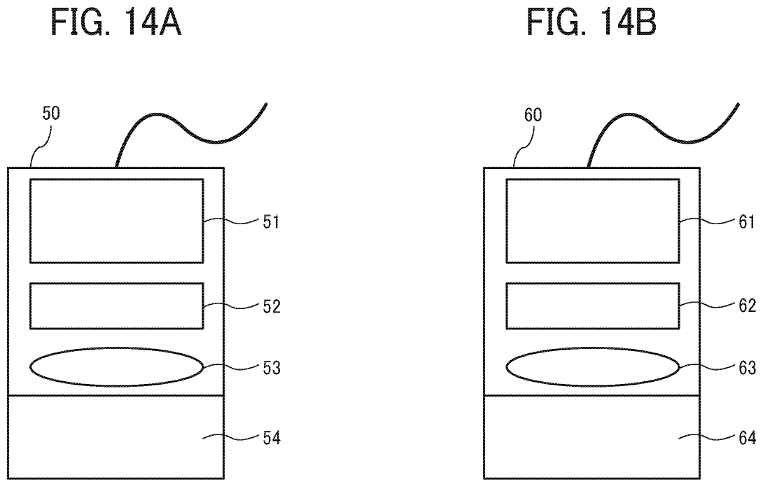

FIGS. 14A and 14B are diagrams of the light projection means and the light reception means of the TD-NIRS system 1 of the present embodiment.

The light source module 50 serving as a light projection means includes, for example, an optical element 53, a VCSEL 52, and an analog circuit 51. FIG. 14A is a diagram of the light source module 50. In the light source module 50, a VCSEL is used as a light emitting element, and the oscillation wavelength of the VCSEL is, for example, 780 nm or 900 nm. The wavelength is selected because the absorption coefficient greatly changes depending on the oxygen concentration in the blood. The analog circuit 51 is a driver for controlling the light source and outputs a drive signal for driving the VCSEL 52 based on a drive instruction from the control unit. Although the light source module may not include the optical element 53, it is preferable that the optical element 53 condenses the light beam output from the VCSEL 52 and irradiates the subject with the light beam to enter the light beam inside the subject.

The light source module 50 is connected to the control unit by wiring. The wiring may be an individual wiring or an I2C wiring in which a clock signal line and a data signal line are bundled.

The optical element 53 of the light source module, the VCSEL 52, and the analog circuit 51 are disposed inside a housing having a window 54. In the present embodiment, these components are accommodated in one housing, but these components may be accommodated in multiple housings by connecting the optical element 53 and the VCSEL 52 with an optical fiber or connecting the VCSEL 52 and the analog circuit 51 with a wiring.

The window 54 is formed by, for example, a transparent resin with respect to the wavelength used. The window 54 is in contact with the subject to be measured (subject to be inspected), and the light condensed in one direction by the optical element 53 passes through the window 54 and enters the surface of the subject.

A transparent gel may be interposed between the subject and the window 54 to enhance contact stability.

A light source module 50 irradiates substantially the same position of the subject with multiple non-parallel light beams. In the present case, the multiple non-parallel light beams with which the light source module 50 irradiates the same position of the subject (scattering body) have different incident angles to the subject and propagate in different propagation paths.

The light detection module 60 serving as a light reception means includes, for example, an optical element 63, a detector 62, and an digital circuit 61 and has a function to measure the reception time. FIG. 14B is a diagram of the light detection module 60.

The light detection module 60 is connected to the control unit by wiring and has a configuration for detecting light emitted in one direction from the light source module 50 and scattered by the subject.

In the present embodiment, the optical element 63, the detector 62, and the digital circuit 61 of the optical detection module are accommodated in one housing, but the optical element 63, the detector 62, and the digital circuit 61 may be accommodated by multiple housing by connecting the optical element 63 and the light detector with an optical fiber or wiring the detector 62 and the digital circuit 61.

The housing is formed by, for example, a light-shielding material (e.g., black resin), and a contact member 64 in contact with the subject is provided at an end portion of the housing. The contact member 64 is formed by, for example, an elastic body. In order to increase the light shielding property, the contact member may include black rubber.

An opening (aperture) is formed in the front end of the housing and the contact member 64. The aperture is a circular opening having a diameter of, for example, about 1 mm, which penetrates the front end of the housing and the contact member 64.

The aperture has a function of limiting the position of the light that propagates through the subject, exits from the subject, and enters the light detection module 60. In the light detection module 60, the contact stability with the subject may be increased by interposing a transparent gel between the contact member 64 and the subject.

Although the light source module may not include the optical element, it is preferable that the optical element 63 such as a lens or diffractive optical element, condenses the light output from the VCSEL 52 and emits the light into the subject. When light enters the light reception element, a photocurrent corresponding to the amount of light flow. The photocurrent is detected by a digital circuit 61 that is a digital circuit, and an electric signal is supplied from the wiring to the control section.

In the TD-NIRS signal, the light intensity and the arrival time change due to scattering and absorption of the light signal from the light projection means by the measurement object (subject to be inspected). Thus, the light reception means includes a time digital converter (TDC) circuit as a function of accurately measuring ToF in the control section.

Since the signal generated by the light detection module 60 changes in the order of several tens of picoseconds, and the signal processing unit 42 may be integrated with the digital circuit 61 of the light detection module 60 because of time accuracy. In order to prevent a signal delay caused by a parasitic resistance due to wiring and in a parasitic capacitance component, it is preferable to arrange the signal processing unit and light detection module on the same substrate, avoid long wiring, and place the signal processing unit and light detection module close to each other.

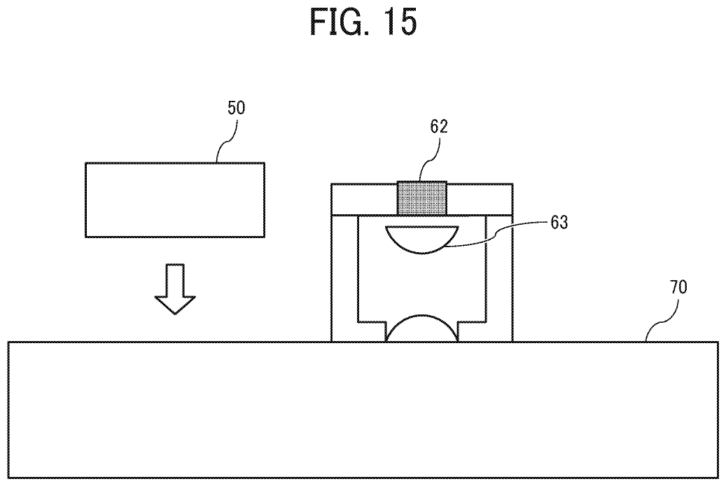

FIG. 15 is a diagram of a configuration of the detector as an example. The light beam output from the light source module 50 is incident on an irradiation spot of the measurement target (subject), passes through the path inside the subject, and is incident on an optical element of the light detection module 60. The incident light is condensed on the detector by the optical element.

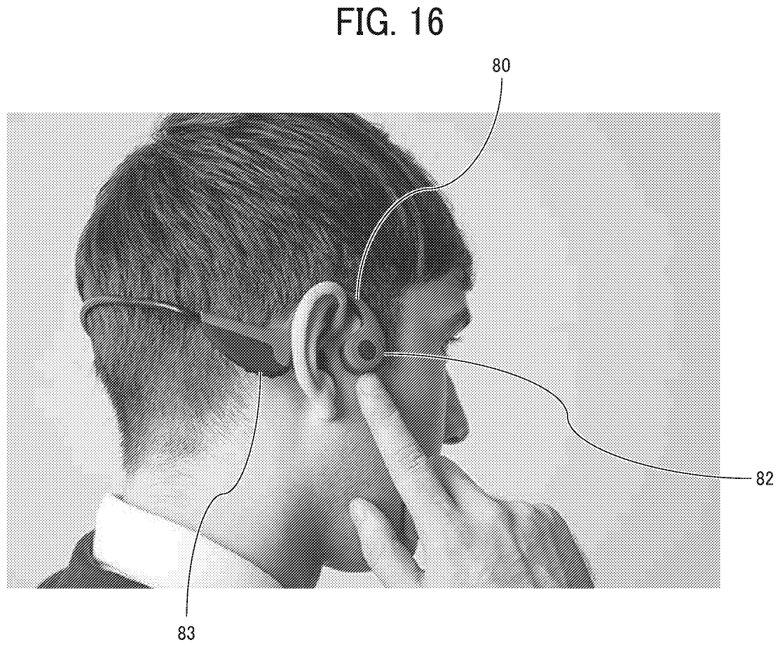

FIG. 16 is a diagram of an ear-hanging TD-NIRS apparatus which is an example of a wearable terminal according to the present embodiment. However, FIG. 16 is a schematic illustration conceptually illustrating the structure of the wearable terminal, and main components are only illustrated for the sake of convenience.

As described above, the wearable terminal is an ear-hanging terminal that is attached to the “ear” of a subject, which is a part of the human body. As illustrated in FIG. 16, the wearable terminal includes a housing 80 having a mounting mechanism. Similar to the TD-NIRS system 1, the housing accommodates a light-emission-and-reception module 83 including a light projection means and a light reception means. The housing also accommodates a control module 82. The control module 82 accommodates a control unit 41, a signal processing unit 42, and a signal transmission unit 43. Alternatively, the signal processing unit 42 may not be disposed in the housing, but may be configured by an external information processing apparatus so as to make the terminal compact.

It is preferable to use a SPAD sensor as the light reception means. As described above, a sensor with high sensitivity and high time resolution is preferable, but the SPAD sensor has high sensitivity and high time resolution. Moreover, the SPAD sensor can integrate large number of pixels and is suitable for small-sized wearable terminals.

In some embodiments, in the measurement system, the detector includes a single photon avalanche diode sensor including one or multiple pixels.

In some embodiments, in the measurement system, the detector detects the multiple scattering light beams corresponding to the pulse light beams, and the circuitry performs a time-resolved spectroscopy measurement based on the detection signal.

The housing is detachably mounted to the front and rear of the ear of the subject by the mounting mechanism. Preferably, the light-emission-and-reception module 83 is mounted so as to be located behind the ear. In the present embodiment, it is assumed that the user usually wears a wearable terminal. Thus, it is preferable that the housing is mounted relatively firmly so that the housing does not come off even if the subject moves actively, and is relatively lightweight so that the housing does not burden the subject. Further, it is preferable that the wearable terminal has a waterproof function.

The wearable terminal is worn while being hooked on the subject's ear. Since the wearable terminal is smaller and lighter, the subject also wear glasses or sunglasses.

In the present embodiment, the subject wears the wearable terminal on one of the right ear or the left ear (the right ear in the example of FIG. 16). However, the wearable terminal may be configured as a common housing for the right ear and the left ear.

Further, the subject may wear two wearable terminals in both ears (right ear and left ear). In such a case, two wearable terminals communicate with each other to cooperate with each other (work together) and has a function equivalent to a function of one wearable terminal.

Preferably, the wearable terminal is driven by a battery. Thus, in the present embodiment, the wearable terminal has a storage battery and works using electric energy stored in the storage battery.

In the case of two channels for both ears, by extracting a signal common to each channel, a deeper part (i.e., a deep part of the brain such as hippocampus, Brodmann area 25 (BA25), or cingulate gyrus) can be measured with much higher accuracy.

As an application of the TD-NIRS system 1 in the present embodiment, a blood flow measurement system will be described below. In the present embodiment, the signal processing unit 42 in the TD-NIRS system 1 performs information processing based on the obtained optical physical values of the subject to be measured. As an example, blood flow measurement, particularly brain blood flow measurement, is performed based on the obtained optical physical values (scattering coefficient μs′ or absorption coefficient μa). The target medium is the amount of brain blood flow in the human body. In such a case, since the scattering coefficient μs′ does not change much, the change of the brain blood flow can be detected by measuring the change of the blood hemoglobin concentration from the variation of the absorption coefficient μa.

In some embodiments, in the measurement system, the circuitry calculates: a scattering coefficient and an absorption coefficient based on the profile, and an amount of hemoglobin in the subject based on the scattering coefficient and the absorption coefficient.

In some embodiments, in the measurement system, the circuitry measures an amount of blood flow in the subject based on a time variation of the amount of hemoglobin.

Further, in the case of TD-NIRS, since ToF based on the average optical path length can be measured, the degree of depth in the brain in which the brain blood flow is changed can be determined. Since the surface emitting laser according to the present embodiment is used, a shallow position and a deep position are easily determined. In particular, there are facial muscles on the surface of the skull in the vicinity of the ear, which causes an artifact. The signal at the shallow position and the signal inside the brain can be determined, and more accurate measurement can be performed.

A first modification of the TD-NIRS system is a measurement system to measure blood flow in muscle. In the present modification, the target medium is, for example, the amount of blood flow in muscle. As a components, an adhesive part to closely contact with the subject.

Activity of the muscle can be confirmed by measuring the amount of the blood flow, and the measurement system can help progress diagnosis of rehabilitation.

In particular, as an application example of a function of separating the position of muscles for each propagation path by using the surface emitting laser according to the present embodiment, determination of the ischemic region of compartment syndrome will be described below.

Compartment syndrome is caused by external wounds and is a state in which internal pressure of a compartment closed by fascia increases, and anomalous venous return in microvessel occurs. In the symptom, ischemia of muscle locally occurs. In the current circumstances, the part is determined by measurement of, for example, internal pressure. However, such a measurement may have a difficulty in the determination and cause an error in a position to be cut open for treatment.

Since an ischemic state can be determined by observing a change in oxygen saturation of hemoglobin in blood, a local ischemic state can be determined from spectral information obtained by NTRS with two wavelengths. In particular, the use of the surface emitting laser of the present embodiment has an advantage in that the light propagation path can be discriminated. For example, in a compartment syndrome of the forearm, the compartments are volar, dorsal, and radial. The volar is divided into a shallow layer and a deep layer.

It is useful to quantitatively determine which part of these four compartments is ischemic. When determining the compartment of the forearm, if the diameter of the forearm is about 80 mm, the light source module 50 and the light detection module 60 can be disposed at positions opposed to the circumference of the forearm for detection.

The transmission light beam can be used by arranging the subject to be examined so that the light source module 50 and the light detection module 60 sandwich the subject and face each other. In the transmission light, since entire transmission light to be detected traverses the forearm, the scattering light with little information from near the surface of the skin can be reduced. When an arrangement of a reflection type is used, the reflection light from the deep position is buried by the scattering light from the surface of the skin. In the present modification, since it is a transmission type, only the scattering light inside the muscle can be detected, and the diagnosis accuracy is increased.

The difference between a shorter propagation path in the diameter direction and a longer propagation path that is bypassed can be determined as time course. In other words, it can be distinguished from the spectral information whether the ischemic region is in the diametrical direction or the bypassed direction. The subject is scanned with one or both of the light source module 50 and the light detection module 60 from the palm side to the radial side while maintaining the opposed arrangement sandwiching the subject. By accumulating and comparing the data while scanning, the ischemic area can be identified.

A second modification of the TD-NIRS system is a measurement system to detect a foreign substance in the living body and cancer. In the second modification, the TD-NIRS is applied to cancer screening. The target medium is the amount of blood flow in the thorax. Measuring the amount of blood flow can help detect breast cancer. Since the blood flow around cancer cells is larger than the blood flow of normal cells, the presence or absence of breast cancer can be determined by measuring the change in the blood flow depending on the breast region.

The optical sensor according to the present embodiment can detect whether there is a foreign substance, for example, cancer, or not. Multiple light source modules 50 and light detection modules 60 are arranged on the surface of the skin at predetermined light transmission-and-reception intervals. Alternatively, by scanning the surface of the skin with the light source module 50 and the light detection module 60, a region different from other regions can be detected.

In some embodiments, in the measurement system, the circuitry detects a foreign substance in the subject based on the detection signal.

In some embodiments, in the measurement system, the circuitry detects a cancer in a living body based on the detection signal.

A living body is irradiated with multiple non-parallel light beams, and light beams from multiple directions are detected by the light detection module 60 to obtain a light quantity ratio. Since the average optical path length is known as described above, information on the degree of the depth of the cancer region from the skin can be obtained.

By adopting the surface emitting laser according to the present embodiment, detection can be performed by separating a deep position and a shallow position. In the case of determining whether an anticancer drug for breast cancer is applicable or not, for which research has become popular in recent years, an ischemic state of cancer cells after administration of the anticancer drug is detected. In such a case, the determination of the ischemic state of the cancer at the deep position has less accuracy when there are many artifacts such as strong ischemia at the shallow portion near the skin due to the anticancer agent.

A typical NIRS has a difficulty in such an analysis in a depth direction. In the present modification, since the deep portion and the shallow portion can be separated at a level of several tens of millimeters, the accuracy of determining whether an anticancer agent is applied to or not is increased by combining the information with position specifying data of the cancer by an ultrasonic device.

A third modification of the TD-NIRS system is a measurement system to estimate a mental state of a person. The mental state of a person is, for example, emotion or attention level. In the present modification, the target medium is the amount of blood flow in the brain, and a mental state can be estimated from the activation site. A close relation between a variation of the amount of blood flow or a blood component such as hemoglobin and neural activity of a person is known.

In some embodiments, in the measurement system, the circuitry estimates a mental state of a person based on the detection signal.

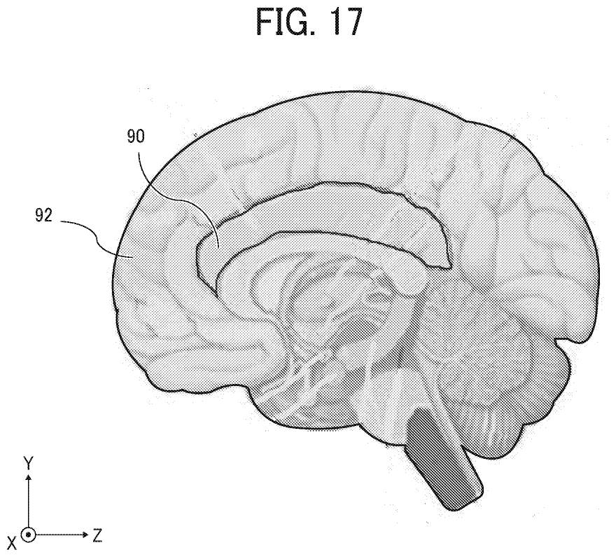

In particular, in the present modification, the amount of blood flow of the cingulate gyrus in the deep part of the brain is detected. FIG. 17 is a cross-sectional image of a brain structure of a person. In FIG. 17, the −Z direction is the direction of the human face. A light source module 50 and a light detection module 60 are installed in the forehead portion. The projection light propagates to the +Z-direction. The distance from the forehead to the cingulate gyrus 90 is approximately 40 to 50 mm for an adult male. Since there is a tissue of the cerebral cortex of the prefrontal cortex 91 such as DLPEC before the cingulate gyrus 90, a typical NTRS has a difficulty in separation between a variation of the amount of blood flow in the shallow position of the tissue and a variation of the amount of blood flow at the cingulate gyrus in a deep position.

As described above, the depth information of the measurement site from the average optical path length can be obtained by using the surface emitting laser according to the present embodiment, and moreover, the minute signal waveform attenuated due to the long average optical path length from the deep portion can be obtained by the SPAD sensor. As a result, the amount of blood flow in the deep portion of the brain can be obtained.

Regions located deep in the brain, such as the hippocampus, the Brodmann area 25 (BA25), and the cingulate gyrus, are activated when stress is sensed, so negative emotion can be estimated.

In particular, it is preferable that cingulate gyrus and DLPFC are separately detected. Since the DLPFC is located at a shallow region as compared with a position of the cingulate gyrus, a typical NTRS has a difficulty in independently detecting the cingulate gyrus. The cingulate gyrus at a deep position inside the DLPFC can be independently measured by using the surface emitting laser according to the present embodiment. It is known that the DLPFC is activated when controlling stress.

Thus, in a case where the cingulate gyrus is activated, and the DLPFC is also activated, the subject controls stress. As described above, when emotion of a person is analyzed, it is useful to correctly determine whether the emotion that the person has is original emotion or controlled emotion.

As described above, in order to analyze the emotion correctly the DLPFC measurement and the cingulate gyrus measurement are separately performed at the same time. The cingulate gyrus at a deep position inside the DLPFC can be independently measured by using the surface emitting laser according to the present embodiment.

As described above, since the activity of nerve cells changes in response to changes in human emotion, the amount of brain blood flow or components in blood changes. Thus, if biological information such as changes in the amount brain blood flow or blood components can be measured, the mental state of a user can be estimated. The user's mental state includes, for example, mood, emotion, health condition, or thermal sensation.

The mood may include, for example, a mood of pleasure or discomfort. Emotions may include, for example, feelings of relief, anxiety, sadness, or resentment. The state of health may include, for example, a state of well-being or weariness. Thermal sensation may include, for example, a sensation of being hot, cold, or muggy.

From the examples described above, indices representing the degree of brain activity, such as proficiency, skill level, and attention level can be similarly estimated. The signal processing circuit may estimate the mental state of the user, such as the attention level, based on the change in the amount of brain blood flow in the measurement unit and output a signal indicating the estimated state.

The blood flow estimation apparatus according to the present embodiment can be applied to a measurement device that acquires internal information of a user in a non-contact manner. The blood flow estimation device according to the present embodiment can be applied to biological or medical sensing, sensing of a driver in an automobile, sensing of a user in a game machine or an attraction device, sensing of a learner in an educational institution, and sensing of a worker in a workplace.

As a fourth modification of the TD-NIRS system, a measurement system for blood circulation diagnosis of the dermis layer is described. After about 100 psec from the peak in decreasing, since the light intensity in the projection light waveform is reduced to 1/e², a distance of several mm as an optical path length can be separated. As a structure of the skin, a layer referred to as a dermis skin layer has a thickness of about 2 mm. There are muscle and fat under the dermal layer. Quantification of the facial skin is useful for aesthetics. There are many cosmetics which aim to increase blood circulation of skin including, for example, scalp, and quantification of the effect of the cosmetics is useful for aesthetics. In the case of skin, the term “blood circulation” is typically used. However, herein, “blood circulation” is used in the same meaning as blood flow.

By using the surface emitting laser according to the present embodiment, the blood circulation state of the skin can be separately detected without being affected by the muscle or fat inside. The blood circulation state of the dermis layer can be accurately and quantitatively detected. For example, the blood circulation state of the dermis can be used as a quantitative indicator for comparison when selecting a personal beauty essence.

In addition, there are various diseases of the skin, and the blood circulation state of the dermis can be also used for quantification of the degree of the various diseases. For example, when determining the degree of a bedsore or burn, a physician determines the degree of depth in which poor blood circulation occurs.

Typically, judgment is made by visual observation, but the condition of the invisible dermis layer can be only judged from the epidermis exposed on the surface. By separating the reception light waveform according to the present embodiment with time course, the blood circulation state of the dermis layer having less influence on the epidermal layer can be quantified.

In particular, since the projection light waveform according to the present embodiment is a short pulse having a peak intensity of 1/e² or less after the lapse of 100 psec from the peak of the light intensity without any skirts, the waveform after 100 psec of the received light waveform can be captured with high accuracy. As a result, a portion before 100 psec in which the effect of the epidermal layer appears and a portion after 200 psec in which the dermis layer appears can be accurately separated. As a result, the blood circulation state of the dermis layer which is not recognized by visual observation can be quantified, and a comprehensive judgment can be made.

As a fifth modification of the TD-NIRS system, the examination of eyeballs using the TD-NIRS system will be described as an example. The eyeball includes is a transparent body referred to as the vitreous body. Many techniques for examining various symptoms in the retina in the back of the vitreous body have been developed. In particular, retinal 3D measurement by optical coherence tomography (OCT) is effective for treating retinal detachment and retinal thinning symptoms.

However, OCT does not observe various symptoms related to the inside of the vitreous body of the transparent body. In principle of OCT, coherence of reflected light is maintained. However, reflected light from a foreign substance inside the vitreous body includes a strong scattering component, and the coherent state is not maintained.

By contrast, by using ToF of the detection principle used in the present embodiment, the distance can be detected even if the scattering light is included. Quantification of the location of the foreign substance present in the vitreous body, such as hemorrhage, is useful in determining subsequent treatment methods. By using the ToF described in the present embodiment, a position in which the foreign substance exists from the surface in the transparent body can be detected.

As described above, an aspect of an embodiment of the present invention is as follows. In a first aspect, a light source device includes; a surface emitting laser to emit a light beam to a subject of a scattering body or a transparent body; and light source control unit to control the surface emitting laser to drive, the surface emitting laser having a light emission having a time width of 200 picoseconds or less in which a light intensity becomes 1/e² with respect to a peak of the light intensity. According to the embodiment of the present invention, a short pulse light source that can preferably measure the inside of the subject of a scattering body or a transparent body can be provided.

In a second aspect, in the light source device according to the first aspect, the surface emitting laser has an light emission having a light intensity of 1/100 or less with respect to the peak of the light intensity after one nanosecond of the peak. According to the embodiment of the present invention, a short pulse light source that can preferably measure the inside of the subject of a scattering body or a transparent body can be provided.

In a third aspect, in the light source device according to the first aspect or the second aspect, the surface emitting laser includes: an active layer; multiple reflectors putting the active layer between the multiple reflectors opposed to each other; and a pair of electrodes to connect a power supply and configured to inject current into the active layer. The laser source control unit has: a current injection period in which current is injected into the active layer; and a current reduction period in which a value of the current injected into the active layer is reduced after the current injection period, laser oscillation does not occur in the current injection period, and laser oscillation occurs in the current reduction period. According to the embodiment of the present invention, a short pulse light source that can preferably measure the inside of the subject of a scattering body or a transparent body can be provided.

In a fourth aspect, in the light source device according to any one of the first aspect to the third aspect, the light surface emitting laser includes: a high refractive region having a high refractive index; and a low refractive region surrounding the high refractive region and having a low refractive index lower than the high refractive index of the high refractive region, the low refractive region is formed by oxidation confinement, the high refractive region has a thickness of 35 nm or less, the low refractive region has a thickness twice or less of the thickness of the high refractive region at a position of three micrometers from a tip of a boundary between the low refractive region and the high refractive region. According to the embodiment of the present invention, a short pulse light source that can preferably measure the inside of the subject of a scattering body or a transparent body can be provided.

In a fifth aspect, a measurement system includes: a light source device including: a surface emitting laser; and a light source control unit to control the surface emitting laser to drive, the light source device to emit a pulse light beam multiple times inside a subject as multiple pulse light beams; and a detector to detect multiple scattering light beams propagating inside the subject and output a detection signal including information on a detection timing; and a signal processing unit to be input the detection signal. The pulse laser beam has a time width of 200 nanoseconds or less in which a light intensity becomes 1/e² with respect to a peak of the light intensity. According to the embodiment of the present invention, a measurement system that can preferably measure the inside of the subject of a scattering body or a transparent body can be provided.

In a sixth aspect, in the measurement system according to fifth aspect, an light intensity after one nanosecond after the peak is 1/100 or less with respect to an intensity of the peak in the light emission of the surface emitting laser. According to the embodiment of the present invention, a measurement system that can preferably measure the inside of the subject of a scattering body or a transparent body can be provided.

In a seventh aspect, in the measurement system according to the fifth aspect or the sixth aspect, the surface emitting laser includes: an active layer; multiple reflectors putting the active layer between the multiple reflectors opposed to each other; and a pair of electrodes to connect a power supply and inject current into the active layer. The laser source control unit has: a current injection period in which current is injected into the active layer; and a current reduction period in which a value of the current injected into the active layer is reduced after the current injection period, laser oscillation does not occur in the current injection period, and laser oscillation occurs in the current reduction period. According to the embodiment of the present invention, a measurement system that can preferably measure the inside of the subject of a scattering body or a transparent body can be provided.

In an eighth aspect, in the measurement system according to any one of the fifth aspect to seventh aspect, the detector includes a SPAD sensor including one or multiple pixels. According to the embodiment of the present invention, a measurement system that can preferably measure the inside of the subject of a scattering body or a transparent body can be provided.

In a ninth aspect, in the measurement system according to any one of the fifth aspect to the eighth aspect, the detector detects the multiple scattering light beams corresponding to the multiple pulse light beams, and the signal processing unit performs a time-resolved spectroscopy measurement based on the detection signal. According to the embodiment of the present invention, a measurement system that can preferably measure the inside of the subject of a scattering body or a transparent body can be provided.

In a tenth aspect, in the measurement system according to any one of the fifth aspect to the ninth aspect, the detector repeatedly detects an incident timing of a single photon, and the signal processing unit generates a profile of an incident timing and a detection frequency. According to the embodiment of the present invention, a measurement system that can preferably measure the inside of the subject of a scattering body or a transparent body can be provided.

In an eleventh aspect, in the measurement system according to any one of the fifth aspect to the tenth aspect, the signal processing unit calculates a scattering coefficient and an absorption coefficient based on the profile, and an amount of hemoglobin in the subject based on the scattering coefficient and the absorption coefficient. According to the embodiment of the present invention, a measurement system suitable for biological measurement can be provided.

In a twelfth aspect, in the measurement system according to the eleventh aspect, the signal processing unit measures an amount of blood flow in the subject based on a time variation of the amount of hemoglobin. According to the embodiment of the present invention, a measurement system suitable for biological measurement can be provided.

In a thirteenth aspect, in the measurement system according to any one of the fifth aspect to the twelfth aspect, the signal processing unit detects a foreign substance in a subject based on the detection signal. According to the embodiment of the present invention, a measurement system that can detect a lesion at an early stage can be provided.

In a fourteenth aspect, in the measurement system according to the thirteenth aspect, the foreign substance is a cancer in a living body. According to the embodiment of the present invention, a measurement system that can detect a lesion at an early stage can be provided.

In a fifteenth aspect, in the measurement system according to any one of the fifth aspect to the twelfth aspect, the signal processing unit estimates a mental state of a person based on the detection signal. According to the embodiment of the present invention, a measurement system that can maintain the health of a subject can be provided.

In a sixteenth aspect, a light source device includes; a surface emitting laser to emit a light beam to a subject; and circuitry to drive the surface emitting laser to emit a light having a time width, a light intensity of which is 1/e² with respect to a peak of the light intensity, of 200 picoseconds or less.

In a seventeenth aspect, in the light source device according to the sixteenth aspect, the surface emitting laser emits the light having a light intensity of 1/100 or less with respect to the peak after one nanosecond of the peak.

In an eighteenth aspect, in the light source device according to the sixteenth aspect or the seventeenth aspect, the surface emitting laser includes: an active layer; multiple reflectors sandwiching the active layer between the multiple reflectors; and an electrode pair connected to a power supply to inject current into the active layer. The circuitry generates a drive signal having: a current injection period in which current is injected into the active layer; and a current reduction period in which a value of the current injected into the active layer is reduced after the current injection period, and the surface emitting laser does not oscillate in the current injection period, and the surface emitting laser oscillates in the current reduction period.

In a nineteenth aspect, in the light source device according to any one of the sixteenth aspect to the eighteenth aspect, the surface emitting laser includes: a first refractive region having a first refractive index; and a second refractive region surrounding the first refractive region and having a second refractive index lower than the first refractive index of the first refractive region, the second refractive region includes an oxide confinement, the first refractive region has a thickness of 35 nm or less, and the second refractive region has a thickness twice or less of the thickness of the first refractive region at a position three micrometers from a tip of a boundary between the second refractive region and the first refractive region.

In a twentieth aspect, a measurement system includes: the light source device according to the sixteenth aspect; a detector to detect multiple scattering light beams propagating inside the subject and output a detection signal including information of a detection timing, the circuitry to: input and process the detection signal; and control the surface emitting laser to emit pulse light beams to an interior of the subject multiple times, and the pulse light beam having a time width, a light intensity of which is 1/e² with respect to the peak, of 200 picoseconds or less. In a twenty-first aspect, in the measurement system according to the twentieth aspect, the surface emitting laser emits the pulse light beams having a light intensity of 1/100 or less with respect to the peak after one nanosecond after the peak.

In a twenty-second, in the measurement system according to the twentieth aspect or the twenty-first aspect, the surface emitting laser includes: an active layer; multiple reflectors sandwiching the active layer between the multiple reflectors; and an electrode pair connected a power supply to inject current into the active layer.

The circuitry generates a drive signal has: a current injection period in which current is injected into the active layer; and a current reduction period in which a value of the current injected into the active layer is reduced after the current injection period, and the surface emitting laser does not oscillate in the current injection period, and the surface emitting laser oscillates in the current reduction period.

In a twenty-third aspect, in the measurement system according to any one of the twentieth aspect to the twenty-second aspect, the detector includes a single photon avalanche diode sensor including one or multiple pixels.

In a twenty-fourth aspect, in the measurement system according to any one of the twentieth aspect to the twenty-third aspect, the detector detects the multiple scattering light beams corresponding to the pulse light beams, and the circuitry performs a time-resolved spectroscopy measurement based on the detection signal.

In a twenty-fifth aspect, in the measurement system according to any one of the twentieth aspect to the twenty-fourth aspect, the detector repeatedly detects an incident timing of a single photon, and the circuitry generates a profile of an incident timing and a detection frequency based on the detection signal.

In a twenty-sixth aspect, in the measurement system according to any one of the twentieth aspect to the twenty-fifth aspect, the circuitry calculates: a scattering coefficient and an absorption coefficient based on the profile, and an amount of hemoglobin in the subject based on the scattering coefficient and the absorption coefficient.

In a twenty-seventh aspect, in the measurement system according to the twenty-sixth aspect, the circuitry measures an amount of blood flow in the subject based on a time variation of the amount of hemoglobin.

In a twenty-eighth aspect, in the measurement system according to the twentieth aspect or the twenty-first aspect, the circuitry detects a foreign substance in the subject based on the detection signal.

In a twenty-ninth aspect, in the measurement system according to the twenty-eighth aspect, the circuitry detects a cancer in a living body based on the detection signal.

In a thirtieth aspect, in the measurement system according to any one of the twentieth aspect to the twenty-seventh aspect, the circuitry estimates a mental state of a person based on the detection signal.

The above-described embodiments are illustrative and do not limit the present invention.

Thus, numerous additional modifications and variations are possible in light of the above teachings. For example, elements and/or features of different illustrative embodiments may be combined with each other and/or substituted for each other within the scope of the present invention.

Any one of the above-described operations may be performed in various other ways, for example, in an order different from the one described above.

The present invention can be implemented in any convenient form, for example using dedicated hardware, or a mixture of dedicated hardware and software. The present invention may be implemented as computer software implemented by one or more networked processing apparatuses. The processing apparatuses include any suitably programmed apparatuses such as a general purpose computer, a personal digital assistant, a Wireless Application Protocol (WAP) or third-generation (3G)-compliant mobile telephone, and so on. Since the present invention can be implemented as software, each and every aspect of the present invention thus encompasses computer software implementable on a programmable device. The computer software can be provided to the programmable device using any conventional carrier medium (carrier means). The carrier medium includes a transient carrier medium such as an electrical, optical, microwave, acoustic or radio frequency signal carrying the computer code. An example of such a transient medium is a Transmission Control Protocol/Internet Protocol (TCP/IP) signal carrying computer code over an IP network, such as the Internet. The carrier medium also includes a storage medium for storing processor readable code such as a floppy disk, a hard disk, a compact disc read-only memory (CD-ROM), a magnetic tape device, or a solid state memory device.

Each of the functions of the described embodiments may be implemented by one or more processing circuits or circuitry. Processing circuitry includes a programmed processor, as a processor includes circuitry. A processing circuit also includes devices such as an application specific integrated circuit (ASIC), a digital signal processor (DSP), a field programmable gate array (FPGA), and conventional circuit components arranged to perform the recited functions.

This patent application is based on and claims priority to Japanese Patent Application No. 2022-121208, filed on Jul. 29, 2022, in the Japan Patent Office, and Japanese Patent Application No. 2023-096438, filed on Jun. 12, 2023, in the Japan Patent Office, the entire disclosure of which are hereby incorporated by reference herein.

### REFERENCE SIGNS LIST

10 Projection light waveform
10M Projection light waveform (ideal form)
10R Projection light waveform (measurement example)
101 Time width of projection light waveform
101H Time width of projection light waveform (FWHM)
101M Time width of projection light waveform (1/e² pulse width)
101L Time for reducing to a peak intensity to 1/1,000
20 Reception light waveform
21 Inclination portion of reception light waveform
22 Time width of reception light waveform
20S Reception light waveform (signal from shallow part)
20D Reception light waveform (signal from deep part)
30 Light source control unit
31 Power supply
32 Driver power supply
33 VCSEL power supply
34 SPAD power supply
35 Circuitry Power supply
40 Circuitry
41 Control unit
42 Signal Processing unit
43 Signal transmission unit
44 Memory unit
50 Light source module
51 Analog circuit (driver)
52 Surface emitting laser
53 Optical element
54 Window portion
60 Light detection module
61 Digital circuitry (DSP)
62 Detector (SPAD Sensor)
63 Optical element
64 Contact member
70 Target medium
70S Light propagation Path (Shallow Position)
70D Light propagation Path (Deep Position)
80 Housing
83 Light emission and reception module
82 Control module
90 Cingulate gyrus
91 Prefrontal area
100 Surface emitting laser
110 n-type GaAs substrate
120 n-type DBR
130 Active layer
140 p-type DBR
150 Oxidation confinement layer
151 Oxidation region
152 Non-oxidation region
153 Outer region
154 Inner region
160 Upper electrode
170 Lower electrode

1. A light source device comprising:
a surface emitting laser to emit a light beam to a subject; and
circuitry configured to drive the surface emitting laser to emit a light having a time width, a light intensity of which is 1/e² with respect to a peak of the light intensity, of 200 picoseconds or less.

2. The light source device according to claim 1, wherein:
the surface emitting laser emits the light having a light intensity of 1/100 or less with respect to the peak after one nanosecond of the peak.

3. The light source device according to claim 1,
wherein the surface emitting laser includes:
an active layer;
multiple reflectors sandwiching the active layer between the multiple reflectors; and
an electrode pair connected to a power supply to inject current into the active layer,
wherein the circuitry generates a drive signal having:
a current injection period in which current is injected into the active layer; and
a current reduction period in which a value of the current injected into the active layer is reduced after the current injection period,
wherein:
the surface emitting laser does not oscillate in the current injection period, and
the surface emitting laser oscillates in the current reduction period.

4. The light source device according to claim 1,
wherein the surface emitting laser includes:
a first refractive region having a first refractive index; and
a second refractive region surrounding the first refractive region and having a second refractive index lower than the first refractive index of the first refractive region,
wherein:
the second refractive region includes an oxide confinement,
the first refractive region has a thickness of 35 nm or less, and
the second refractive region has a thickness twice or less of the thickness of the first refractive region at a position three micrometers from a tip of a boundary between the second refractive region and the first refractive region.

5. A measurement system comprising:
the light source device according to claim 1; and
a detector to detect multiple scattering light beams propagating inside the subject and output a detection signal including information of a detection timing,
wherein the circuitry is further configured to:
input and process the detection signal; and
control the surface emitting laser to emit pulse light beams to an interior of the subject multiple times,
wherein the pulse light beam includes a time width, a light intensity of which is 1/e² with respect to the peak, of 200 picoseconds or less.

6. The measurement system according to claim 5, wherein:
the surface emitting laser emits the pulse light beams having a light intensity of 1/100 or less with respect to the peak after one nanosecond after the peak.

7. The measurement system according to claim 5,
wherein the surface emitting laser includes:
an active layer;
multiple reflectors sandwiching the active layer between the multiple reflectors; and
an electrode pair connected a power supply to inject current into the active layer,
wherein the circuitry generates a drive signal having:
a current injection period in which current is injected into the active layer; and
a current reduction period in which a value of the current injected into the active layer is reduced after the current injection period,
wherein:
the surface emitting laser does not oscillate in the current injection period, and
the surface emitting laser oscillates in the current reduction period.

8. The measurement system according to claim 5, wherein:
the detector includes a single photon avalanche diode sensor including one or multiple pixels.

9. The measurement system according to claim 5, wherein:
the detector detects the multiple scattering light beams corresponding to the pulse light beams, and
the circuitry performs a time-resolved spectroscopy measurement based on the detection signal.

10. The measurement system according to claim 5, wherein:
the detector repeatedly detects an incident timing of a single photon, and
the circuitry generates a profile of an incident timing and a detection frequency based on the detection signal.

11. The measurement system according to claim 10, wherein the circuitry calculates:
a scattering coefficient and an absorption coefficient based on the profile, and
an amount of hemoglobin in the subject based on the scattering coefficient and the absorption coefficient.

12. The measurement system according to claim 11, wherein:
the circuitry measures an amount of blood flow in the subject based on a time variation of the amount of hemoglobin.

13. The measurement system according to claim 5, wherein:
the circuitry detects a foreign substance in the subject based on the detection signal.

14. The measurement system according to claim 13, wherein:
the circuitry detects a cancer in a living body based on the detection signal.

15. The measurement system according to claim 5, wherein:
the circuitry estimates a mental state of a person based on the detection signal.
